(19) United States
(12) Patent Application Publication
(10) Pub. No.: US 2014/0011809 A1
(43) Pub. Date: Jan. 9, 2014

(54) DRUG DELIVERY TO THE ANTERIOR AND POSTERIOR SEGMENTS OF THE EYE USING EYE DROPS

(71) Applicant: Raouf Rekik, Montplaisir (TN)

(72) Inventor: Raouf Rekik, Montplaisir (TN)

(21) Appl. No.: 13/912,896

(22) Filed: Jun. 7, 2013

Related U.S. Application Data
(62) Division of application No. 12/921,958, filed on Jan. 13, 2011.

(30) Foreign Application Priority Data
Mar. 11, 2008 (TN) SN08110

(51) Int. Cl. A61K 31/5377; A61K 31/4168; A61K 31/502; A61K 31/403; A61K 31/137

(52) U.S. Cl. CPC A61K 31/5377; A61K 31/403; A61K 31/137; A61K 31/502; A61K 31/4168
USPC 514/236.2; 514/412; 514/653; 514/249; 514/392

(57) ABSTRACT

A method and means for delivery of drugs to the chorio-retina and the optic nerve head which comprises contacting the surface of the eye with an effective amount of drug for treatment of chorio-retina and optic nerve head and a physiologically acceptable adrenergic agent for enhancing delivery of the drug to these tissues in an ophtalmologically acceptable carrier, said adrenergic agent being selected from the group consisting of alpha adrenergic agonist agents, derivatives of the alpha adrenergic agonist agents, beta-blocking agents, derivatives of the beta-blocking agents and mixtures thereof.

### FIELD OF THE INVENTION

The present invention relates to a method to deliver drugs to the anterior and posterior segments of the eye and compositions thereof for such a delivery. More specifically the present invention relates to a method and composition wherein an alpha adrenergic agonist agent, a beta-blocking agent, a derivative or derivatives thereof, or mixture thereof is (are) administered, with an effective amount of a drug that can treat chorio-retinal and/or optic nerve head disorders. Compositions containing said alpha adrenergic agonist agent, beta-blocking agent, derivative or derivatives thereof, or mixture thereof and the drug are also disclosed,

### BACKGROUND OF THE INVENTION AND RELATED PRIOR ART

With the population living longer many disorders or diseases of the eyes have been appearing and are currently being treated by ophthalmologists. Over the past several years many advances in ophthalmic therapy have arisen in response to a rising need for improvement in this area.

Many ophthalmic disorders arise in the anterior and posterior segments of the eye. The anterior segment of the eye is the front third of the eye that includes the structures in front of the vitreous humor such as the cornea, iris, ciliary body and lens. Ophthalmic disorders associated with the anterior segment of the eye include glaucoma, cataract, congenital and developmental abnormalities, inflammatory and infectious diseases, hereditary and degenerative diseases and ocular manifestations of systemic diseases, tumors, injury and trauma.

The posterior segment of the eye is the back two-thirds of the eye that includes the anterior hyaloid membrane and all of the structures behind it such as the vitreous humor, the retina, the choroid and the optic nerve. Opthalmic disorders resulting in the posterior segment of the eye include age-related macular degeneration, diabetic retinopathy, retinal venous occlusions, retinal arterial occlusions, macular edema, post-operative inflammation, uveitis, retinitis, proliferative vitreoretinopathy, glaucoma neuropathy and high myopia macular degeneration.

Administering drugs for treatment of various disorders of the eye can be performed by a variety of methods known in the art such as by topical administration, administration of eye drops, intraocular injection and systemic administration.

However most of these methods have drawbacks. Eye drops and ointments that have been used for years are not always effective due to the eye's natural protective surface. Furthermore, by using this type of administration the drugs are rarely delivered to the posterior segments of the eye in proper quantities and hence cannot be used to treat various disorders and/or diseases of the chorio-retinal and/or optic nerve head disorders.

Systemic administration requires a very high dosage of drug and like topical administration very little of the administered compound enters the eye.

For treating disorders and/or diseases of the chorio-retina and/or optic nerve head the main type of administration utilized is injecting the various drugs directly into the eye, usually into the vitreous humor or subconjunctival injections. For example ranibizumab (Lucentis®) injection is known for treating wet age-related macular degeneration. Ranibizumab is a vascular endothelial growth factor (VEGF) antagonist that blocks abnormal blood vessel growth and leakage in the eye. It must be administered once a month by a physician.

Pegatanib (Macugen®) and bevacizumab (Avastin®) are other drugs for treating age-related macular degeneration. They must be delivered directly by injection through a needle into the eye, which requires medical assistance and is usually performed in a clinical setting.

In the cases of delivering the drugs by direct injection into the eye, usually only one eye is treated at a time to prevent complications and these techniques are invasive techniques that are often very discomforting to the patient. In some instances direct injection can lead to complications in the eyes that are even more serious than the disease or disorder itself.

Due to the membrane barriers of the cornea, conjunctiva and sclera and lachrymal drainage it is quite difficult to administer successfully drugs into the posterior segment of the eye other than by injection.

In general, drugs can enter the eye through three distinct routes; i.e., (1) the corneal route which is through the anterior chamber and then through the lens, the pupil or the iris; (2) the conjunctival route which either is directly across the sclera, choroid, choriocapillaries and retinal pigment epithelium to the retina or indirectly into the retrobulbar space and then the optic nerve head; and (3) from the systemic circulation after topical, parental, oral or intranasal or any other route that delivers the drug to the blood circulation.

The use of eye drops is generally a route of delivering drugs into the posterior segment of the eyes which is considered quite ineffective due to the lack of therapeutic amounts of the drugs that can be effective in this area of the eye. See, for example Myles et al Adv. Drug Del Rev. 57(14)2063-79). Due to this ineffectiveness, several attempts have been made to overcome this problem in the art.

Thus, U.S. Patent publication No. 2007/0020336 A1 describes the use of cyclodextrin nanotechnology for delivery to the posterior segment of the eye. This ophthalmic composition contains a drug, cyclodextrin and water in which about 0.1% to 90% (w/v) of the drug is dissolved in the solution and a solid phase consisting of particles which have a size of 10 nm to 1 mm. This composition can be in the form of eye drops.

In yet another attempt to solve the problem with non-invasive posterior segment delivery of drugs U.S. publication No. 2005/0009910 A1 describes a method and composition which uses an effective concentration of an ester prodrug of the active drug. This composition is a sustained release composition in which a polymeric microparticle system enhances the release of the drug. The prodrug is administered via injection or implantation.

WO 2007/075720 A2 describes topical mecamylamine formulations for ocular administrations for the treatment of neovascularization, abnormal angiogenesis, vascular permeability or combinations thereof of posterior and/or anterior tissues and fluids of the eye.

Although the art has advanced quite rapidly in the last few years, there is still a need in this art to provide methods and compositions to deliver drugs to treat various ophthalmic disorders and/or diseases to the posterior segments of the eyes which drug is delivered through a non-invasive route.

Thus it is an object of the present invention to overcome the problems associated with drug delivery to the posterior segment of the eye known in the art.

It is another object of the present invention to provide a method for delivering drugs to treat ophthalmic disorders or diseases that uses drugs known in the art to treat these disorders and/or diseases.

Yet another object of the present invention is to provide a non-invasive treatment for delivering drugs to treat ophthalmic disorders or diseases which is simple and less expensive.

Another object of the present invention is to provide an ophthalmic composition comprising an effective amount of a drug to treat the ophthalmic disorder and/or disease and an adrenergic agent selected from the group consisting of alpha adrenergic agonist agents, derivatives of the alpha adrenergic agonist agents, beta-blocking agents, derivatives of the beta-blocking agents and mixtures thereof, in an effective amount such that the drug used to treat the ophthalmic disorders and/or diseases of the eye is delivered to the posterior segment of the eye.

In yet another embodiment the present invention provides a method to deliver drugs to the chorio-retina and optic nerve head of the eye.

Use of an adrenergic agent selected from the group consisting of alpha adrenergic agonist agents, derivatives of the alpha adrenergic agonist agents, beta-blocking agents, derivatives of the beta-blocking agents and mixtures thereof, to deliver drugs to treat ophthalmic disorders and/or diseases is also part of the present invention.

These and other objects are achieved by the present invention as evidenced by the summary of the invention, description of the preferred embodiments and the claims.

### SUMMARY OF THE INVENTION

Thus, the present invention relates to a method for delivering drugs to the posterior and anterior segments of the eyes comprising: contacting the surface of the eye with an effective amount of a drug for treating eye disorders and/or diseases of the eye and a physiologically acceptable amount of an adrenergic agent selected from the group consisting of alpha adrenergic agonist agents, derivatives of the alpha adrenergic agonist agents, beta-blocking agents, derivatives of the beta-blocking agents and mixtures thereof, in an ophtalmologically acceptable carrier.

In another embodiment, the present invention relates to a method of treating a eye disorders and/or diseases of the eye by delivering drugs to the chorio-retina and optic nerve head of an eye comprising administering to a person or an animal in need of such treatment an effective amount of a drug for treatment of the chorio-retina and optic nerve head and a physiologically acceptable amount of an adrenergic agent selected from the group consisting of alpha adrenergic agonist agents, derivatives of the alpha adrenergic agonist agents, beta-blocking agents, derivatives of the beta-blocking agents and mixtures thereof.

The present invention relates to polynucleotides enabling the rapid, simple and specific detection of Group B Streptococcus highly-virulent ST-17 clones.

a) an adrenergic agent selected from the group consisting of alpha adrenergic agonist agents, derivatives of the alpha adrenergic agonist agents, beta-blocking agents, derivatives of the beta-blocking agents and mixtures thereof, and

b) a drug for treating eye disorders and/or diseases of the eye.
In yet another embodiment, the present invention provides a composition comprising, consisting or consisting essentially of

a) an adrenergic agent selected from the group consisting of alpha adrenergic agonist agents, derivatives of the alpha adrenergic agonist agents, beta-blocking agents, derivatives of the beta-blocking agents and mixtures thereof; and

b) a drug that treats disorders and/or diseases of the eyes.

In yet another embodiment the present invention provides a method for increasing the transfer of a drug into the eye orbit, the posterior sclera and then into chorio-retina and optic nerve head to treat disorders and/or diseases of the eye comprising contacting the surface of an eye or both eye(s) with a physiologically acceptable amount of an adrenergic agent selected from the group consisting of alpha adrenergic agonist agents, derivatives of the alpha adrenergic agonist agents, beta-blocking agents, derivatives of the beta-blocking agents and mixtures thereof, and a pharmaceutically acceptable amount of a second drug that treats disorders and/or diseases of the eyes.

An adrenergic agent for the transfer, to the posterior segment of one or both eye(s), of a second drug that treats disorders and/or diseases of the eyes, to treat diseases and/or disorders of the eye is another aspect of the present invention.

In yet another embodiment the present invention provides a kit comprising or consisting of:

a) an adrenergic agent selected from the group consisting of alpha adrenergic agonist agents, derivatives of the alpha adrenergic agonist agents, beta-blocking agents, derivatives of the beta-blocking agents and mixtures thereof or a composition comprising or consisting of said adrenergic agent; and

b) a drug that treats disorders and/or diseases of the eyes.

The present invention also provides use of

a) an adrenergic agent selected from the group consisting of alpha adrenergic agonist agents, derivatives of the alpha adrenergic agonist agents, beta-blocking agents, derivatives of the beta-blocking agents and mixtures thereof; and

b) a pharmaceutically acceptable amount of a second drug that treats disorders and/or diseases of the eyes

for the manufacture of a medicament to treat disorders and/or diseases of the eye.

### BRIEF DESCRIPTION OF THE DRAWINGS

FIG. 1 is a photograph of an ocular fundus fluorescence of the right and left eyes of a patient who had apraclonidine and fluorescein administered in the right eye (A) and fluorescein in the left eye (B).

FIG. 2 is a photograph of an ocular fundus fluorescence of the right and left eyes of a patient who had neosynephrine and fluorescein administered in the right eye (A) and fluorescein in the left eye (B).

FIG. 3 is a photograph of an ocular fundus fluorescence of the right and left eyes of a patient who had fluorescein administered in the right eye (A) and timolol and fluorescein in the left eye (B).

FIG. 4 is a photograph of an ocular fundus fluorescence of the right and left eyes of a patient who had fluorescein administered in the right eye (A) and apraclonodin and fluorescein in the left eye (B).

FIG. 5 is a photograph of an ocular fundus fluorescence of the right and left eyes of a patient who had fluorescein administered in the right eye (A) and brimonidine (Alphagan) and fluorescein in the left eye (B).

FIG. 6 is a photograph of an ocular fundus and a fluoro-angiography of a diabetic patient (patient B.C.N.) who received topically a combination of apraclonidine and bevacizumab (Avastin®).

FIG. 7 is an optical coherence tomography scan (O.C.T.) of the same diabetic patient as the one of FIG. 6 (patient B.C.N) prior to treatment (A), and two months after treatment (B) with apraclonidine and bevacizumab (Avastin®).

FIG. 8 is a photograph of an ocular fundus and fluoro-angiography of a patient (patient G.N.) having non proliferative diabetic retinopathy before she receives topically a combination of a corticosteroid, a non-steroidal anti-inflammatory agent and apraclonidine.

FIG. 9 is an optical coherence tomography scan (O.C.T.) of the same patient as the one of FIG. 8 (patient G.N.) prior to treatment (A) and three months after treatment (B) with a combination of a corticosteroid, a non-steroidal anti-inflammatory agent and apraclonidine.

FIG. 10 is a photograph of an ocular fundus and fluoro-angiography of a patient (patient M.M.) having non proliferative diabetic retinopathy before she receives topically a combination of a corticosteroid and neosynephrine.

FIG. 11 is an optical coherence tomography scan (O.C.T.) of the same patient as the one of FIG. 10 (patient M.M.) prior to treatment (A) and three months after treatment (B) with a corticosteroid and neosynephrine.

FIG. 12 is a photograph of an ocular fundus and fluoro-angiography of a patient (patient H.M.) having proliferative diabetic retinopathy before (A) and 3 months after (B) he receives a combination of brimonidine and a non-steroidal anti-inflammatory agent.

FIG. 13 is an optical coherence tomography scan (O.C.T.) of the same patient as the one of FIG. 12 (patient H.M.) prior to treatment (A) and three months after treatment (B) with a combination of brimonidine and a non-steroidal anti-inflammatory agent.

FIG. 14 is a photograph of an ocular fundus and fluoro-angiography of a patient (patient N.B.) having non proliferative diabetic retinopathy before (A) and six months after (B) having received topically a combination of a corticosteroid, a non-steroidal anti-inflammatory agent and brimonidine.

FIG. 15 is an optical coherence tomography scan (O.C.T.) of the same patient as the one of FIG. 14 (patient N.B.) prior to treatment (A) and six months after treatment (B) with a combination of a corticosteroid, a non-steroidal anti-inflammatory agent and brimonidine.

FIG. 16 is a photograph of an ocular fundus and fluoro-angiography of a patient (patient M.R.) having diabetic retinopathy before he receives topically a combination of apraclonidine and bevacizumab (Avastin®).

FIG. 17 is an optical coherence tomography scan (O.C.T.) of the same patient as the one of FIG. 16 (patient M.R.) prior to treatment (A) and three months after treatment (B) with apraclonidine and bevacizumab (Avastin®).

FIG. 18 is a photograph of an ocular fundus and fluoro-angiography of a patient (patient N.B.) having non proliferative diabetic retinopathy and branched vein occlusion in the left eye before (A) and three months after (B) having received topically a combination of a corticosteroid, a non-steroidal anti-inflammatory and neosynephrine.

FIG. 19 is an optical coherence tomography scan (O.C.T.) of the same patient as the one of FIG. 18 (patient N.B.) prior to topical treatment (A) and three months after topical treatment (B) with a combination of a corticosteroid, a non-steroidal anti-inflammatory and neosynephrine.

FIG. 20 is a photograph of an ocular fundus and a fluoro-angiography of a patient (patient B.S.T.) having imminent central retinal vein occlusion in the left eye before (A) and two weeks after (B) having received topically a combination of a non-steroidal anti-inflammatory and apraclonidine.

FIG. 21 is an optical coherence tomography scan (O.C.T.) of the same patient as the one of FIG. 20 (patient B.S.T.) prior to (A) and eye two weeks after (B) topical treatment with a combination of a non-steroidal anti-inflammatory and apraclonidine.

FIG. 22 is a photograph of an ocular fundus and fluoro-angiography of a patient (patient A.H.) having central retinal vein occlusion in the left eye before (A) and three months after (B) having received topically a combination of a corticosteroid and a non-steroidal anti-inflammatory and neosynephrine.

FIG. 23 is an optical coherence tomography scan (O.C.T.) of the same patient as the one of FIG. 22 (patient A.H.) prior to topical treatment (A) and three months after topical treatment (B) with a combination of a corticosteroid, a non-steroidal anti-inflammatory and neosynephrine.

FIG. 24 is an optical coherence tomography scan (O.C.T.) of a patient (patient S.Z.) having branched retinal vein occlusion in the left eye prior to topical treatment (A) and three months after topical treatment (B) with a combination of ramipril and apracronidine.

FIG. 25 is a photograph of a fluoro-angiography (A) and an ocular fundus (B) and of a patient (patient D.M.H) having branched retinal occlusion in the right eye before he receives topically a combination of bevacizumab (Avastin®) and neosynephrine.

FIG. 26 is an optical coherence tomography scan (O.C.T.) of the same patient as the one of FIG. 25 (patient D.M.H.) prior to topical treatment (A) and one month after topical treatment (B) with a combination of bevacizumab (Avastin®) and neosynephrine.

FIG. 27 is a photograph of an ocular fundus (upper line) and a fluoro-angiography (lower line) of a patient (patient CH.A.) having a two-month history of decreased vision (because of age related macular degeneration) in the right eye before (A) and two months after (B) having received topically a combination of a corticosteroid, a non-steroidal anti-inflammatory agent and apraclonidine.

FIG. 28 is an optical coherence tomography scan (O.C.T.) of the same patient as the one of FIG. 27 (patient CH.A.) prior to topical treatment (A) and two months after topical treatment (B) with a combination of a corticosteroid, a non-steroidal anti-inflammatory agent and apraclonidine.

FIG. 29 is a photograph of an ocular fundus (upper line) and fluoro-angiography (lower line) of a patient (patient K.T.) having a three-month history of occult new vessel (age related macular degeneration) in both eyes before (A) and three months after (B) having received topically a combination of a corticosteroid, a non-steroidal anti-inflammatory agent and neosynephrine.

FIG. 30 is an optical coherence tomography scan (O.C.T.) of the same patient as the one of FIG. 29 (patient K.T.) prior to treatment (A) and three months after treatment (B) with a combination of a corticosteroid, a non-steroidal anti-inflammatory agent and neosynephrine.

FIG. 31 is a photograph of an ocular fundus (upper line) and fluoro-angiography (lower line) of a patient (patient D.F.) having a history of decreased vision in the left eye because of choroid occult new vessel (age related macular degeneration), before (A) and three months after (B) having received topically a combination of a corticosteroid, a non-steroidal anti-inflammatory agent and apraclonidine.

FIG. 32 is an optical coherence tomography scan (O.C.T.) of the same patient as the one of FIG. 31 (patient D.F.) prior to treatment (A) and three months after treatment (B) with a combination of a corticosteroid, a non-steroidal anti-inflammatory agent and apraclonidine.

FIG. 33 is an optical coherence tomography scan (O.C.T.) of a patient (patient D.M.) having a history of decreased vision in the right eye because of choroid occult new vessel (age related macular degeneration), prior to treatment (A) and one month after treatment (B) with a combination of bevacizumab (Avastin®) and apraclonidine.

FIG. 34 is a photograph of an ocular fundus and fluoro-angiography of a patient (patient B.S.S.) having complaints of decreased central vision in the right eye before treatment (A), two months after having received topically a combination of bevacizumab (Avastin®) and apraclonidine (B), four months after having stopped the topical treatment (C) and two weeks after readministration of bevacizumab and apraclonidine (D).

FIG. 35 is a vertical scan of an optical coherence tomography scan (O.C.T.) of a patient (patient G.C.) having complaints of decreased central vision in the right eye due to classic macular new vessels prior to treatment (A) and one week after treatment (B) with ramipril, timolol and neosynephrine.

FIG. 36 is a horizontal scan of an optical coherence tomography scan (O.C.T.) of the same patient as the one of FIG. 35 (patient G.C.) prior to treatment (A) and one week after treatment (B) with ramipril, timolol and neosynephrine.

FIG. 37 are results from field of vision tests from a patient (patient A.L.) having an open angle chronic glaucoma in the right eye prior to topical treatment (A) and two weeks after topical treatment (B) with ramipril and brimonidine.

### DETAILED DESCRIPTION OF THE PREFERRED EMBODIMENTS OF THE PRESENT INVENTION

As used herein the terminology “anterior segment of the eye” means the front third of the eye that includes the structures in front of the vitreous humor such as the cornea, iris, ciliary body and lens.

As used herein the terminology “posterior segments of the eye” means the back two-thirds of the eye that includes the anterior hyaloid membrane and the optical structures behind it such as the vitreous humor, retina, choroid, optic nerve and optic nerve head.

The term “optic nerve head” as used herein means the circular area in the back (posterior segment) of the eye where the optic nerve connects to the retina.

As used herein the term “chorio-retina” refers to the posterior segments of the eye in which the retina contacts the choroid, which is the middle membrane of the eye.

The terms “treating” and “treatment” mean that the eye disorder and/or eye disease is improved.

The term “eye disorders” encompasses changes in vision, in the appearance of the eye or having abnormal sensations in the eye. Eye disorders include optic nerve disorders, chorio-retinal disorders and trauma such as injuries to the eye.

As used herein, the term “eye diseases” means any disease of the eye such as of glaucomatous neuropathy, central serous chorio retinopathy, high myopia chorio-retinopathy, pigmentosa retinopathy, diabetic retinopathy, central retinal vein occlusion, branch retinal vein occlusion, presbyopia, age related vision degradation, central retinal artery occlusion, exsudative macular degeneration, uveitis, papillitis and endophthalmitis. This terminology also encompasses at least one of the above diseases and thus two or more diseases of the above diseases, of the eye are also contemplated by this expression. In a particular embodiment, “eye diseases” refer to diseases that affect the posterior segment of the eye.

By “eye disorders and/or diseases of the eye”, it is meant herein at least one eye disorder and/or disease of the eye; this term can encompass several (two, three or more than three) eye disorders and/or diseases of the eye.

The term “animal” includes mammalians, in particular humans and non human mammalians. The term “mammal” encompasses any of various warm-blooded vertebrate animals of the class Mammalia, including humans and non human mammalians, characterized by a covering of hair on the skin and, in the female, milk-producing mammary glands for nourishing the young.

As used herein “ophthalmologically acceptable carrier” means any carrier that has substantially no long term or permanent detrimental effect on the eye to which it is administered, in particular any carrier that can be placed in the eye and that does not cause eye irritation. Opthamologically acceptable carriers include water (distilled or deionized water), saline solutions, phosphate buffered saline solutions, and other aqueous media.

The term “adrenergic agent” as used herein encompasses an alpha adrenergic agonist agent, a derivative of an alpha adrenergic agonist agent, a beta-blocking agent, a derivative of a beta-blocking agents and mixtures thereof.

As used herein, an “alpha adrenergic agonist agent” is a drug which has effects similar to, or the same as, epinephrine (adrenaline) or which is susceptible to epinephrine, or similar substances, such as biological receptors. This term includes alpha 1 agonists, and alpha 2 agonists. Alpha 1 agonists stimulate phospholipase C activity in a human and an animal body, which results in vasoconstriction and mydriasis (excessive dilation of the pupil). Alpha 2 agonists are able to inhibit adenyl cyclase activity in a human and an animal body and are used notably as antihypertensives, sedatives, to reduce eye's aqueous humor secretions and to facilitate aqueous humor outflow via the uveoscleral route. Examples of alpha 1 agonist include neosynephrine. Examples of alpha 2 agonists include brimonidine, aprachlonidine and clonidine. Others alpha adrenergic agonist agents that can be used in the present methods and compounds of the present invention include methoxamine, methylnorepinephrine, oxymetazoline, phenylephrine, neosynephrine pivalat, beta-methylepinephrine, guanfacine, guanabenz, guanoxabenz, guanethidine, tizanidine, and mixtures thereof.

By “beta-blocking agent” (or beta-adrenergic antagonist agent) it is meant herein a drug which blocks the action of epinephrine (adrenaline) and/or norepinephrine (noradrenaline) in a human and an animal body. These compounds are used notably to lower intraocular tension and/or to reduce eye's aqueous humor secretions. This term encompasses antagonists of the beta 1, beta 2 and beta 3 adrenergic receptors. The beta-blocking agents that can be used in the methods, the compositions and the kits of the present invention include timolol, sotalol, propranolol, penbutolol, nadolol, metoprolol, labetalol, esmolol, carteolol, bisoprolol, betaxolol, atenolol, acebutolol, levobunolol, metipranolol and mixtures thereof.

By “derivative of an alpha adrenergic agonist agent”, it is meant a compound obtained via a chemical modification of an alpha 1 agonist or an alpha 2 agonist, and which retains respectively the ability to stimulate phospholipase C activity or the ability to inhibit adenyl cyclase activity in an animal model such as a mouse, a rat or a monkey. Said derivatives are preferably amine-containing compounds, which more preferably have pKa's of greater than 7, preferably about 7.5 to 9. The alpha 1 or alpha 2 activity of a derivative of an adrenergic agonist agent can be shown for example, by applying, to one eye of a mouse, a rat or a monkey, few drops (one, two or three) of said derivative in solution in an ophthalmologically acceptable carrier, and applying, to the other eye of the same animal, the same volume of the ophthalmologically acceptable carrier alone, and comparing dilation of the pupil (in the case of an alpha 1 agonist derivative) or aqueous humor secretions (in the case of an alpha 2 agonist derivative) of both eyes. “Derivatives of an alpha adrenergic agonist agent” include imidazoline derivatives such as oxymetazoline, xylometazoline, tetrahydrozoline and the like. Also those derivatives defined in U.S. Pat. Nos. 7,345,077 and 7,335,803 can also be used as derivatives in the methods, compositions and kits of the present invention.

By “beta-blocking agent derivative”, it is meant a compound obtained via a chemical modification of a beta-blocking agent as defined above, and which retains the ability to lower intraocular tension and/or to reduce eye's aqueous humor secretions in an animal model such as a mouse, a rat or a monkey. These properties can be shown for example, by applying, to one eye of a mouse, a rat or a monkey, few drops (one, two or three) of said derivative in solution in an ophthalmologically acceptable carrier, and applying, to the other eye of the same animal, the same volume of the ophthalmologically acceptable carrier alone, and measuring and comparing intraocular tension and/or aqueous humor secretions of both eyes. Beta-blocking agent derivatives include guaiacoxy propanolamine derivatives such as those described in U.S. Pat. No. 5,804,603.

Thus, the present invention relates to a method for delivering drugs to the posterior and anterior segments of the eyes (of one or both eye(s)) comprising contacting the surface of the eye with an effective amount of a drug for treating eye disorders and/or diseases of the eye and a physiologically acceptable amount of an adrenergic agent selected from the group consisting of alpha adrenergic agonist agents, derivatives of the alpha adrenergic agonist agents, beta-blocking agents, derivatives of the beta-blocking agents and mixtures thereof, in an opthalmologically acceptable carrier.

Accordingly, the methods, compositions and kits of the present invention provide for the treatment of many eye disorders and/or diseases of the eye such as glaucoma, glaucoma neuropathy, diabetic retinopathy, choroidal new vessels (age-related macular degeneration; high myopia; macular degeneration), uveitis (in particular anterior and/or posterior uveitis), eye infections, papillitis, endophthalmitis, optic nerve head inflammation, arterial or vein occlusion, central serous chorio-retinopathy (CSCR), pigmentosa retinopathy. The methods, compositions and kits of the present invention can in particular be used for improving vision of one or both eyes, and more particularly for improving distance vision and/or near vision. In addition, the methods, compositions and kits of the present invention can be used to perform topic anaesthesia as a prelude to surgery.

At (east one eye disorder and/or disease of the eye can be treated with the methods and compositions of the present invention and more than one or several eye disorders and/or diseases can also be treated.

In the present invention, the drug to treat the disorders and/or diseases of the eyes is administered topically in the form of suspensions, gels or ointments or in the form of eye drops or solutions along with a physiologically acceptable amount of an adrenergic agent selected from the group consisting of alpha adrenergic agonist agents, derivatives of the alpha adrenergic agonist agents, beta-blocking agents, derivatives of the beta-blocking agents and mixtures thereof.

In a particular embodiment, active compounds are administered in accordance with the present invention to the eye admixed with an ophthalmically acceptable carrier. Any suitable, e.g., conventional, ophthalmically acceptable carrier as defined herein may be employed.

It may be desirable to formulate the drug for treating eye disorders and/or diseases of the eye and the adrenergic agent that can be used in the methods, the compositions and the kits of the present invention as topical agents to be instilled into the eye. Such formulations may contain the active ingredient in a concentration range of approximately 0.01% to 20% weight by volume (w/v), preferably from 0.05% to 10% (w/v), and more preferably from 0.5% to 3% (w/v). The composition itself may include, in addition to the active ingredient, excipients which are per se well know in the art for preparing ophthalmic compositions, particularly ophthalmic solutions.

The ophthalmic compositions (solutions or other formulations) that contain the adrenergic agent(s) and/or the drug(s) for treating disorders and/or diseases of the eyes may be administered to the mammalian eye as often as necessary to obtain an improvement of the eye disorder and/or eye disease. Those skilled in the art will recognize that the frequency of administration and duration of treatment depends on the precise nature of the active ingredient(s) and its concentration in the ophthalmic formulation, and various factors such as the type and severity of the eye disorder and/or eye disease, the age and weight of the patient, the patient's general physical condition and the cause of the eye disorder and/or eye disease. Within these guidelines it is contemplated that the ophthalmic formulations (preferably ophthalmic solutions) of the present invention will be administered topically to the mammalian eye approximately once, twice or three times daily. The duration of treatment administered in accordance with the present invention may range, for example, from few weeks (at least one week) to few months (at least one month), in particular from 1 week to 6 months, preferably at least 2 weeks and less than 4 months and more preferably at least 3 weeks and less than 3 months. However, a prolonged treatment may be required. In particular, the treatment may last for life, for example in case of recurrence of the eye disorder and/or eye disease.

In a particular embodiment of the invention, the physiologically acceptable amount of the adrenergic agent(s) is administered prior to administering the effective amount of the drug(s) for treating disorders and/or diseases of the eyes; hence, in this case, the adrenergic agent(s) contact the eye prior to the drug(s) for treating disorders and/or diseases of the eyes.

The alpha adrenergic agonist agents that can be used in the methods, the compositions and the kits of the present invention can be selected from the group comprising or consisting of methoxamine, methylnorepinephrine, oxymetazoline, phenylephrine, neosynephrine, in particular neosynephrine pivalat, beta-methylepinephrine, brimonidine, apraclonidine, clonidine, guanfacine, guanabenz, guanoxabenz, guanethidine, tizanidine, and mixtures thereof.

The beta-blocking agents that can be used in the methods, the compositions and the kits of the present invention can be selected from the group comprising or consisting of timolol, sotalol, propranolol, penbutolol, nadolol, metoprolol, labetalol, esmolol, carteolol, bisoprolol, betaxolol, bisoprolol, atenolol, acebutolol, levobunolol, metipranolol and mixtures thereof.

The actual amount of the adrenergic agent(s) and the drug(s) for treating eye disorders and/or diseases of the eye to be administered in any given case will be determined by a physician taking into account the relevant circumstances, such as the type and severity of the eye disorder and/or eye disease, the age and weight of the patient, the patient's general physical condition and the cause of the eye disorder and/or eye disease.

By way of example, physiologically acceptable amounts of the alpha adrenergic agonist agent, the beta-blocking agent, the derivative(s) of an alpha adrenergic agonist agent, the derivative(s) of a beta-blocking agent and mixtures thereof that are generally administered to a person or an animal in need thereof are ranging from 0.01% to 20% (w/v), preferably from 0.1% to 15% (w/v) and more preferably from 0.1% to 3% (w/v), for example from 0.1% to 2% (w/v) or 0.2% to 1% (w/v) if eyedrops are used and from 0.1% to 2% (w/v) in the case of topical administration.

The effective amount of a drug for treating eye disorders and/or diseases of the eye is generally administered to a person or an animal in need thereof in a concentration ranging from 0.001 to 15% (w/v), preferably from 0.05 to 10% (w/v), and more preferably from 0.1 to 3% (w/v).

The substances or drugs used for treating disorders of the eyes and/or eye diseases can be selected from the group of calcium antagonists, angiotensin converting enzyme inhibitors, nitrates or nitric oxide generators, beta adrenergic agonists, antioxidants and radical scavengers, dopaminergic and serotoninergic agents, monoamine oxidase inhibitors, anti-inflammatory agents, growth factors, neuroprotective agents, growth factor vasoactive agents, neuropeptides, anti-inflammatory mediators, anti-infective agents, anti-ischemic association agents, non-steroidal anti-inflammatory agents, anti-growth factor agents, and mixtures thereof.

At least one drug or substance for treating eye disorders and/or diseases of the eye can be used in the methods, the compositions and the kits of the present invention. For example if a person or an animal has more than one eye disorder and/or eye disease, several drugs can be administered at the same time providing that these drugs do not interact with themselves to provide adverse side effects. Thus, for example, one can administer at least one anti-inflammatory and at least one angiotensin converting enzyme inhibitor.

The drugs or combinations of drugs can be administered at room temperature.

Examples of calcium antagonists that can be used in the methods, the compositions and the kits of the present invention can selected from the group comprising verapamil, nifedipine, nimadipine, diltiazem, nicardipine, felodipine, amlodipine, isradipine and mixtures thereof.

Examples of angiotensin converting enzyme inhibitors that can be used in the methods, the compositions and the kits of the present invention are selected from the group comprising captopril, enalapril, usinopril, ramipril, kinapril, benazepril, cilazapril and mixtures thereof.

Nitrates, isorbide dinitrate, isorbide mononitrate, linsidomine and mixtures thereof are examples of nitrates or nitric oxide generators that can be used in the methods, the compositions and the kits of the present invention.

Beta adrenergic agonists that can be used in the methods, the compositions and the kits of the present invention can be selected from the group comprising salbutamol, terbutalin, isoprenalin and mixtures thereof while antioxidants and radical scavengers that can be used in the present invention can be selected from the group comprising ascorbic acid, glutathione catalases and their derivatives and mixtures thereof.

Dopaminergic and serotoninergic agent that can be used in the methods, the compositions and the kits of the present invention can be selected from the group comprising: levodopa, amantadine, bromocriptine, serotonine and mixtures thereof.

Amitryptiline, nortryptyline, selegiline and mixtures thereof are monoamine oxidase inhibitors that can be used in the methods, the compositions and the kits of the present invention.

Examples of anti-inflammatory agents that can be used in the methods, the compositions and the kits of the present invention are non-steroidal anti-inflammatory agents or steroidal anti-inflammatory agents, in particular corticosteroids, or mixtures thereof.

Examples of non-steroidal anti-inflammatory drugs that can be used in the methods, the compositions and the kits of the present invention can be selected from the group comprising or consisting of aspirin, arylalkanoic acids such as bromfenac, indometacin, oxameticin, 2-aryipropionic acids such as fenbufen, pirprofen, ketoprofen, ibuprofen, oxaprozin, and ketorolac, femamic acids, pyrazolidine derivatives such as clofezonem kebuzone and phenazone, ocicams such as droxicam and meloxicam, and COX-2 inhibitors, such as celecoxib and rofecoxib.

Examples of corticosteroids that can be used in the methods, the compositions and the kits of the present invention can be selected from the group consisting of cortisone, hydrocortisone, deltacortisone or prednisolone, prednisone, deltahydrocortisone or prednisolone, methylprednisolone or medrocortisone, fluorohydrocortisone or fluorocortisone, fluoromethyiprednisolone or dexamethazone, fluoromethyldeltahydrocortisone or betamethazone and paramethazone.

Growth factors such as nerve growth factors (NGF), epidermal growth factor (EGF), platelet derived growth factor (PDGF), transforming growth factor (TGF) and mixtures thereof can be used in the methods, the compositions and the kits of the present invention.

Anti-growth factor agents that can be used in the methods, the compositions and the kits of the present invention include anti-vascular endothelial growth factor (anti-VEGF) agents, anti-insulin like growth factor (anti-IGF) agents, anti-fibroblast growth factor (anti-FGF) agents, anti-platelet derived growth factor (anti-PDGF) agents, anti-placenta growth factor agents and mixtures thereof.

Anti-VEGF agents that can be used in the methods, the compositions and the kits of the present invention can be selected from the group comprising bevacizumab (Avastin®), ranibizumab (Lucentis®), pegaptanib (Macugen®), and mixtures thereof.

Anti-inflammatory mediators that can be used in the methods, the compositions and the kits of the present invention can be selected from the group comprising cytokines, bradikinine, histamine, serotonin, thrombin, ADP, acethylcholine, adrenalin and derivatives and mixtures thereof.

Anti-infective agents that can be used in the methods, the compositions and the kits of the present invention can be selected from the group comprising antibiotics, antifungal agents, antiviral agents and mixtures thereof.

Anti-ischemic association compounds selected from the group comprising angiotensin converting enzyme inhibitors, non-steroidal anti-inflammatory agents and mixtures thereof, can also be used in the methods, the compositions and the kits of the present invention.

In a particular embodiment, the drugs (i.e., the adrenergic agent(s) and the drug(s) to for treating disorders and/or diseases of the eyes) are delivered to the posterior segment of the eye. Said drugs can be in particular delivered to the chorio-retina and optic nerve head of the eyes.

In another embodiment, the present invention relates to a method of treating eye disorders and/or diseases of the eye by delivering drugs to the chorio-retina and optic nerve head of an eye comprising administering to a person or an animal in need of such treatment
an effective amount of a drug for treatment of the chorio-retina and/or optic nerve head and
a physiologically acceptable amount of an adrenergic agent selected from the group consisting of alpha adrenergic agonist agents, derivatives of the alpha adrenergic agonist agents, beta-blocking agents, derivatives of the beta-blocking agents and mixtures thereof.

As set forth above the effective amount of the drug for treating chorio-retina and optic nerve head can be selected among the group comprising calcium antagonists, angiotensin converting enzyme inhibitors, nitrates or nitric oxide generators, beta-adrenergic agonists, antioxidants and radical scavengers, dopaminergic and serotoninergic agents, monoamine oxidase inhibitors, anti-inflammatory agents, growth factors, neuropeptides, anti-inflammatory mediators, anti-infective agents, non-steroidal anti-inflammatory agents, anti-ischemic association agents (non-steroidal anti-inflammatory agents and angiotensin converting enzyme inhibitors), neuroprotective agents, growth factor vasoactive agents, anti-growth factor agents, in particular anti-vascular endothelial growth factor (anti-VEGF) agents, anti-insulin like growth factor (anti-IGF) agents, anti-fibroblast growth factor (anti-FGF) agents, anti-platelet derived growth factor (anti-PDGF) agents, anti-placenta growth factor agents, and mixtures thereof. The specific drugs and amounts that can also be used in this method are set forth above.

The alpha adrenergic agonist agents that can be used in the methods, the compositions and the kits of the present invention can be selected from the group comprising or consisting of methoxamine, methylnorepinephrine, oxymetazoline, phenylephrine, neosynephrine pivalat, beta-methylepinephrine, brimonidine, apraclonidine, clonidine, guanfacine, guanabenz, guanoxabenz, guanethidine, tizanidine, and mixtures thereof.

The beta-blocking agents that can be used in the methods, the compositions and the kits of the present invention can be selected from the group comprising or consisting of timolol, sotalol, propranolol, penbutolol, nadolol, metoprolol, labetalol, esmolol, carteolol, bisoprolol, betaxolol, bisoprolol, atenolol, acebutolol, levobunolol, metipranolol and mixtures thereof.

Accordingly, the effective amount of a drug for treatment of the chorio-retina and/or optic nerve head is administered in a concentration ranging from 0.001 to 15% (w/v), preferably from 0.05 to 10% (w/v), and more preferably from 0.1 to 3% (w/v).

The physiologically acceptable amounts of the alpha adrenergic agonist agent, the beta-blocking agent, the derivative of an alpha adrenergic agonist agent, the derivative of a beta-blocking agent and mixtures thereof that are generally administered are ranging from 0.01% to 20% (w/v), preferably from 0.1% to 15% (w/v), and more preferably from 0.1% to 3% (w/v), for example from 0.1% to 2% (w/v) or 0.2% to 1% (w/v) if eyedrops are used and from 0.1% to 2% (w/v) in the case of topical administration. More specifically, the physiologically acceptable amounts of alpha adrenergic agonist agent(s) or derivatives thereof that are generally administered vary from 0.01% to 20% (w/v), preferably from 0.1 to 3% (w/v) while the physiologically acceptable amounts of beta-blocking agent(s) or derivatives thereof that are generally administered vary from 0.05% to 2% (w/v), preferably 0.1% to 1% (w/v), and more preferably from 0.1% to 0.5% (w/v).

In the method for treating chorio-retina and optic nerve head, the drug(s) and the physiologically acceptable amounts of an adrenergic agent can be administered simultaneously or said adrenergic agent can be administered prior to the drug(s) which are used to treat the at least one eye disorder and/or eye disease.

If said adrenergic agent selected from the group consisting of alpha adrenergic agonist agents, derivatives of the alpha adrenergic agonist agents, beta-blocking agents, derivatives of the beta-blocking agents and mixtures thereof is administered before the drug(s), usually it is administered from 1 second to 3 hours, preferably from 5 to 60 minutes, prior to the administration of the treating drug(s).

In yet another embodiment, the present invention provides a composition comprising, consisting or consisting essentially of

a) an adrenergic agent selected from the group consisting of alpha adrenergic agonist agents, derivatives of the alpha adrenergic agonist agents, beta-blocking agents, derivatives of the beta-blocking agents and mixtures thereof, and

b) a drug that treats eye disorders and/or diseases of the eye.

Said drug for treating eye disorders and/or diseases of the eye can be selected among the group comprising calcium antagonists, angiotensin converting enzyme inhibitors, nitrates or nitric oxide generators, beta-adrenergic agonists, antioxidants and radical scavengers, dopaminergic and serotoninergic agents, monoamine oxidase inhibitors, anti-inflammatory agents, growth factors, neuropeptides, anti-inflammatory mediators, anti-infective agents, non-steroidal anti-inflammatory agents, anti-ischemic association agents (non-steroidal anti-inflammatory agents and angiotensin converting enzyme inhibitors), anti-growth factor agents, in particular anti-vascular endothelial growth factor (anti-VEGF) agents, anti-insulin like growth factor (anti-IGF) agents, anti-fibroblast growth factor (anti-FGF) agents, anti-platelet derived growth factor (anti-PDGF) agents, anti-placenta growth factor agents, and mixtures thereof.

In a particular embodiment, said composition comprises both (i) an alpha adrenergic agonist agents or a derivative thereof and (ii) a beta-blocking agent, or a derivative thereof.

In a particular embodiment, said drug for treating eye disorders and/or diseases of the eye is selected from the group consisting of angiotensin converting enzyme inhibitors, non-steroidal anti-inflammatory agents, anti-growth factor agents, steroidal anti-inflammatory agents, in particular corticosteroids, and mixtures thereof. Said drug(s) are as set forth above.

In another particular embodiment, said drug for treating eye disorders and/or diseases of the eye is an anti-growth factor agent as defined herein, in particular an anti-vascular endothelial growth factor agent (anti-VEGF agent), a corticosteroid as set forth above or mixtures thereof.

In another particular embodiment, said drug for treating eye disorders and/or diseases of the eye is an angiotensin converting enzyme inhibitor and/or a non-steroidal inflammatory agent (anti-ischemic complex). In this case, the adrenergic agent is generally selected from the group consisting of alpha adrenergic agonist agents, derivatives of the alpha adrenergic agonist agents and mixtures thereof. Said drug(s) are as set forth above.

In another particular embodiment, said drug for treating disorders and/or diseases of the eyes is a corticosteroid and/or an anti-VEGF agent. Said drug(s) are as set forth above.

The compositions of the invention can be used as a medicament. In particular, said composition are suitable for the treatment and in particular the topical treatment of a disorder and/or a disease of the eye selected from the group comprising or consisting of diabetic retinopathy, retinal artery and vein occlusion, age related vision degradation (near and far visual acuity; visual field), macular oedema, central serious chorio-retinopathy, exsudative macular degenreration (age related macular degeneration, high myopia; macular degeneration) uveitis, papillitis, glaucomaneuropathy.

The alpha adrenergic agonist agents that can be used in the methods, the compositions and the kits of the present invention can be selected from the group comprising or consisting of methoxamine, methylnorepinephrine, oxymetazoline, phenylephrine, neosynephrine pivalat, beta-methylepinephrine, brimonidine, apraclonidine, clonidine, guanfacine, guanabenz, guanoxabenz, guanethidine, tizanidine, and mixtures thereof.

The beta-blocking agents that can be used in the methods, the compositions and the kits of the present invention can be selected from the group comprising or consisting of timolol, sotalol, propranolol, penbutolol, nadolol, metoprolol, labetalol, esmolol, carteolol, bisoprolol, betaxolol, atenolol, acebutolol, levobunolol, metipranolol and mixtures thereof.

In this composition the angiotensin converting enzyme inhibitors that can be used can be selected from the group comprising or consisting of captopril, enalapril, usinopril, ramipril, kinapril, benazepril, cilazapril and mixtures thereof.

The non-steroidal anti-inflammatory agents that can be used in the composition can be selected from the group comprising or consisting of aspirin, arylalkanoic acids such as bromfenac, indometacin, oxameticin, 2-arylpropionic acids such as fenbufen, pirprofen, ketoprofen, ibuprofen, oxaprozin, and ketorolac, femamic acids, pyrazolidine derivatives such as clofezonem kebuzone and phenazone, ocicams such as droxicam and meloxicam, and COX-2 inhibitors, such as celecoxib and rofecoxib.

In the compositions of the present invention, the adrenergic agent is generally present in amounts ranging from 0.01% to 20% (w/v), preferably from 0.1% to 15% (w/v), and more preferably from 0.1% to 3% (w/v), for example from 0.1% to 3% (w/v), 0.2% to 2% (w/v) or 0.1% to 0.5% (w/v). More specifically, the amounts of alpha adrenergic agonist agent(s) or derivatives thereof that are present in the compositions of the invention generally vary from 0.01% to 20% (w/v), preferably from 0.1% to 3% (w/v) while the amounts of beta-blocking agent(s) or derivatives thereof that are present in the compositions of the invention generally vary from in the amounts of 0.05% to 2% (w/v), preferably 0.1% to 1% (w/v), and more preferably from 0.1% to 0.5% (w/v). The drug for treating eye disorders and/or diseases of the eye is generally present in amounts ranging from 0.001% to 15% (w/v), preferably from 0.05% to 10% (w/v), and more preferably from 0.1% to 3% (w/v).

A composition for the treatment and in particular the topical treatment of a disorder and/or disease of the eye selected from the group comprising of diabetic retinopathy, central retinal vein occlusion, branch retinal vein occlusion, retinal artery occlusion, exsudative macular degeneration (age related macular degeneration; high myopia) uveitis, papillitis, and endophthalmitis comprising, consisting or consisting essentially of

a) an adrenergic agent selected from the group consisting of alpha adrenergic agonist agents, derivatives of the alpha adrenergic agonist agents, beta-blocking agents, derivatives of the beta-blocking agents and mixtures thereof, and

b) an anti-vascular endothelial growth factor (anti-VEGF), corticosteroid(s) or mixture thereof is another embodiment of the present invention.

The alpha adrenergic agonist agents that can be used in the methods, the compositions and the kits of the present invention can be selected from the group comprising or consisting of methoxamine, methylnorepinephrine, oxymetazoline, phenylephrine, neosynephrine, in particular neosynephrine pivalat, beta-methylepinephrine, brimonidine, apraclonidine, clonidine, guanfacine, guanabenz, guanoxabenz, guanethidine, tizanidine, and mixtures thereof.

The beta-blocking agents that can be used in the methods, the compositions and the kits of the present invention can be selected from the group comprising or consisting of timolol, sotalol, propranolol, penbutolol, nadolol, metoprolol, labetalol, esmolol, carteolol, bisoprolol, betaxolol, atenolol, acebutolol, levobunolol, metipranolol and mixtures thereof.

The anti-VEGF is selected from the group comprising or consisting of bevacizumab (Avastin®), ranibizumab (Lucentis®), pegaptanib (Macugen®) and mixtures thereof.

The corticosteroids can be selected from the group consisting of cortisone, hydrocortisone, deltacortisone or prednisolone, prednisone, deitahydrocortisone or prednisolone, methylprednisolone or medrocortisone, fluorohydrocortisone or fluorocortisone, fluoromethylprednisolone or dexamethazone, fluoromethyldeltahydrocortisone or betamethazone and paramethazone.

The amounts of alpha adrenergic agent(s), beta-blocking agent(s), derivative(s) thereof or mixtures thereof that can be used are as set forth above.

The amounts of anti-VEGF and/or corticosteroids that can be used are generally ranging from 0.001% to 15% (w/v), preferably from 0.05% to 10% (w/v), and more preferably from 0.1% to 3% (w/v).

In yet another embodiment the present invention provides a composition for the topical treatment of diabetic retinopathy macular oedema, exudative macular degeneration, central retinal vein occlusion or branch retinal vein occlusion, uveitis, papillitis, or endophtalmitis comprising, consisting or consisting essentially of

a) an adrenergic agent selected from the group consisting of alpha adrenergic agonist agents, derivatives of the alpha adrenergic agonist agents, beta-blocking agents, derivatives of the beta-blocking agents and mixtures thereof, and

b) angiotensin converting enzyme inhibitor(s) and/or non steroidal anti-inflammatory agent(s) and/or steroidal anti-inflammatory agent(s), in particular corticosteroid(s), or mixtures thereof.

The adrenergic agent, angiotensin converting enzyme inhibitor(s), non steroidal anti-inflammatory agent(s) and corticosteroid(s) used in this composition can be any drug set forth above with respect to the other methods and compositions.

The amounts of alpha adrenergic agent(s), beta-blocking agent(s), derivative(s) thereof or mixtures thereof that can be used are as set forth above.

The amounts of angiotensin converting enzyme inhibitor(s), non steroidal anti-inflammatory agent(s) and/or corticosteroid(s) that can be used are generally ranging from 0.001% to 15% (w/v), preferably from 0.05% to 10% (w/v), and more preferably from 0.1% to 3% (w/v).

In yet another embodiment the present invention provides a composition for the topical treatment of age related vision degradation and presbyopia comprising or consisting of:

a) an alpha adrenergic agonist agent and/or a derivative of an alpha adrenergic agonist agent and

b) an angiotensin converting enzyme inhibitor and/or a non steroidal anti-inflammatory agent and/or an anti vascular endothelial growth factor agent (anti-VEGF agent) selected from the group comprising bevacizumab (Avastin®), ranibizumab (Lucentis®), pegaptanib (Macugen®) and mixtures thereof.

The alpha adrenergic agonist agent, angiotensin converting enzyme inhibitor and non steroidal anti-inflammatory agent and corticosteroid(s) used in this composition can be any drug set forth above with respect to the other methods and compositions.

The amounts of alpha adrenergic agent(s), derivative(s) thereof or mixtures thereof that can be, used are as set forth above.

The amounts of angiotensin converting enzyme inhibitor(s), non steroidal anti-inflammatory agent(s) and/or anti-VEGF agent(s) that can be used are generally ranging from 0.001% to 15% (w/v), preferably from 0.05% to 10% (w/v), and more preferably from 0.1% to 3% (w/v).

In yet another embodiment, the present invention provides a method for increasing the transfer of a drug into the eye orbit, the posterior sclera and then into chorio-retina and optic nerve head to treat disorders and/or diseases of the eye comprising contacting the surface of an eye or both eyes with

a) a physiologically acceptable adrenergic agent selected from the group consisting of alpha adrenergic agonist agents, derivatives of the alpha adrenergic agonist agents, beta-blocking agents, derivatives of the beta-blocking agents and mixtures thereof, and

b) a pharmaceutically acceptable amount of a second drug that treats disorders and/or diseases of the eyes.

As set forth above the alpha adrenergic agonist agents that can be used in the methods, the compositions and the kits of the present invention can be selected from the group comprising or consisting of methoxamine, methylnorepinephrine, oxymetazoline, phenylephrine, neosynephrine pivalat, beta-methylepinephrine, brimonidine, apraclonidine, clonidine, guanfacine, guanabenz, guanoxabenz, guanethidine, tizanidine, and mixtures thereof.

The beta-blocking agents that can be used in the methods, the compositions and the kits of the present invention can be selected from the group comprising or consisting of timolol, sotalol, propranolol, penbutolol, nadolol, metoprolol, labetalol, esmolol, carteolol, bisoprolol, betaxolol, atenolol, acebutolol, levobunolol, metipranolol and mixtures thereof.

The amounts of alpha adrenergic agent(s), beta-blocking agent(s), derivative(s) thereof or mixtures thereof that can be used are as set forth above.

The pharmaceutically acceptable amount of a second drug that treats disorders and/or diseases of the eyes that can be used is as set forth above.

An adrenergic agent for the transfer, to the posterior segment of the eyes, of a second drug that treats disorders and/or diseases of the eyes, for the treatment of diseases and/or disorders of the eye is another aspect of the present invention. Said adrenergic agent is selected from the group consisting of alpha adrenergic agonist agents, derivatives of the alpha adrenergic agonist agents, beta-blocking agents, derivatives of the beta-blocking agents and mixtures thereof.

As set forth above the alpha adrenergic agonist agents that can be used in the methods, the compositions and the kits of the present invention can be selected from the group comprising or consisting of methoxamine, methylnorepinephrine, oxymetazoline, phenylephrine, neosynephrine pivalat, beta-methylepinephrine, brimonidine, apraclonidine, clonidine, guanfacine, guanabenz, guanoxabenz, guanethidine, tizanidine, and mixtures thereof.

The beta-blocking agents that can be used in the methods, the compositions and the kits of the present invention can be selected from the group comprising or consisting of timolol, sotalol, propranolol, penbutolol, nadolol, metoprolol, labetalol, esmolol, carteolol, bisoprolol, betaxolol, atenolol, acebutolol, levobunolol, metipranolol and mixtures thereof.

The amounts of alpha adrenergic agent(s), beta-blocking agent(s), derivative(s) thereof or mixtures thereof that can be used are as set forth above.

The second drug that treats, disorders and/or diseases of the eyes that can be used in the methods, the compositions and the kits of the present invention can be selected from the group comprising or consisting of a calcium antagonists, nitrates or nitric oxide generators, beta adrenergic agonists, antioxidants and radical scavengers, dopaminergic and serotoninergic agents, monoamine oxidase inhibitors, anti-inflammatory agents, growth factors, neuropeptides, anti-inflammatory mediators, anti-infective agents, anti-ischemic association agents (non-steroidal anti-inflammatory agents and angiotensin converting enzyme inhibitors), anti-growth factor agents, in particular anti-vascular endothelial growth factor (anti-VEGF) agents, anti-insulin like growth factor (anti-IGF) agents, anti-fibroblast growth factor (anti-FGF) agents, anti-platelet derived growth factor (anti-PDGF) agents, anti-placenta growth factor agents, and mixtures thereof. The specific drugs that can also be used in this method are set forth above. The specific drugs that are used to treat eye disorders and/or diseases can be any drug set forth above with respect to the other methods.

The amounts of alpha adrenergic agent(s), beta-blocking agent(s), derivative(s) thereof or mixtures thereof that can be used are as set forth above.

The pharmaceutically acceptable amount of the second drug depends upon which drug is being used. Examples of pharmaceutically acceptable amounts include amounts ranging from 0.1 to 15% (w/v), preferably from 0.5 to 10%, and more preferably from 0.5 to 3% (w/v).

The present invention also provides use of

a) an adrenergic agent selected from the group consisting of alpha adrenergic agonist agents, derivatives of the alpha adrenergic agonist agents, beta-blocking agents, derivatives of the beta-blocking agents and mixtures thereof, and

b) a pharmaceutically acceptable amount of a second drug that treats disorders and/or diseases of the eyes, for the manufacture of a medicament to treat disorders and/or diseases of the eye.

As set forth above the alpha adrenergic agonist agents that can be used in the methods, the compositions and the kits of the present invention can be selected from the group comprising or consisting of methoxamine, methylnorepinephrine, oxymetazoline, phenylephrine, neosynephrine pivalat, beta-methylepinephrine, brimonidine, apraclonidine, clonidine, guanfacine, guanabenz, guanoxabenz, guanethidine, tizanidine, and mixtures thereof.

The beta-blocking agents that can be used in the methods, the compositions and the kits of the present invention can be selected from the group comprising or consisting of timolol, sotalol, propranolol, penbutolol, nadolol, metoprolol, labetalol, esmolol, carteolol, bisoprolol, betaxolol, atenolol, acebutolol, levobunolol, metipranolol and mixtures thereof.

The amounts of adrenergic agents that are present in this medicament are as set forth above.

The pharmaceutically acceptable amount of a second drug that treats disorders and/or diseases of the eyes that can be used is as set forth above.

In yet another embodiment the present invention provides a kit comprising, consisting or consisting essentially of:
(a) an adrenergic agent selected from the group consisting of alpha adrenergic agonist agents, derivatives of the alpha adrenergic agonist agents, beta-blocking agents, derivatives of the beta-blocking agents and mixtures thereof, or a composition comprising, consisting or consisting essentially of said adrenergic agent, and
(b) a drug that treats disorders and/or diseases of the eyes.

The alpha adrenergic agents, beta-blocking agents, and derivatives thereof, as well as the drug for treating disorders and/or diseases of the eyes are as set forth above.

The amounts of said adrenergic agent and said drug for treating disorders and/or diseases of the eyes that can be used are as set forth above.

These kits can be used in the methods of the present invention, to treat chorio-retinal and/or optic nerve head disorders in a person or an animal.

In a particular embodiment, said kits comprise both (i) an alpha adrenergic agonist agents or a derivative thereof and (ii) a beta-blocking agent, or a derivative thereof.

In a particular embodiment, said drug for treating disorders and/or diseases of the eyes is selected from the group consisting of angiotensin converting enzyme inhibitors, non-steroidal anti-inflammatory agents, anti-growth factor agents, steroidal anti-inflammatory agents, in particular corticosteroids, and mixtures thereof. These drugs are as set forth above.

In a particular embodiment, said drug for treating disorders and/or diseases of the eyes is an angiotensin converting enzyme inhibitor and/or a non-steroidal anti-inflammatory agent (anti-ischemic complex). In this case, the adrenergic agent is generally selected from the group consisting of alpha adrenergic agonist agents, derivatives of the alpha adrenergic agonist agents and mixtures thereof. Said drug(s) are as set forth above.

In another particular embodiment, drug for treating disorders and/or diseases of the eyes is a corticosteroid and/or an anti-VEGF agent. Said drug(s) are as set forth above.

The invention will now be illustrated by the following description of clinical examples which, of course, are not limiting in nature. Further characteristics of the invention will become clear from the following clinical observations that are, of course, provided only by way of illustration and do not in any way limit the scope of the invention.

### EXAMPLES

### Background and Methods Used

There are two general pathways whereby a drug can reach the posterior segment of the eye from an eye drop:

1. Corneal: into the anterior chamber, and then through the lens, the pupil or the iris.
2. Conjunctival: either directly across the sclera, choroid, choriocapillaris and retinal pigment epithelium to the retina, or indirectly into the retrobulbar space and then the ONH (optic nerve head).

There is evidence that mechanically blocking of the corneal surface has little effect on drug penetration into the posterior tissues, which suggests that the conjunctival route is the more important for drug delivery.

When a large drop is allowed to flood the interpalpebal space, the fluid could fall under gravity and distend the cul-de-sac. In that case, the drug would have the opportunity to penetrate into the posterior sclera and orbit. The penetration from a drop to the posterior segment is increased by using an alpha adrenergic agonist agent or a beta-blocking agent as the drug carrier.

Alpha adrenergic agonist agents or beta-blocking agents or both enhance the transfer of drugs to the chorio-retina through the conjunctive route using two steps. The first step is the sequestration of the drug in the orbit because of vasoconstriction. The second step involves the transfer of the drug to the choroid because of oncotic pressure. Alpha adrenergic agents induce tension in the Bruck Wallace muscle and then in the bruck membrane. As a result choroid hydrostatic pressure decreases and oncotic pressure becomes relatively more important. Choroid arteries constriction by alpha adrenergic or beta-blocking agents increases arterial resistance, decreases hydrostatic pressure in capillaries and venules, so that oncotic pressure becomes relatively higher than the hydrostatic pressure, thus increasing the diffusion of the drug for treatment from the orbit to the chorio-retina and optic nerve head.

In the examples below, the following formulations were administered topically to patients:
apraclonidine (Lopidine®; alpha adrenergic agonist agent): 0.5% (w/v);
bevacizumab (Avastin®; anti-VEGF agent): 2% (w/v);
brimonidine (Alphagan®; alpha adrenergic agonist agent): 0.2% (w/v);
dexamethasone (Tobradex®; corticosteroid): 0.1% (w/v);
fluorescein: 10% (w/v);
indomethacin (Indocollyre®; non-steroidal anti-inflammatory agent): 0.1% (w/v);
neosynephrine (alpha adrenergic agonist agent): 10% (w/v);
prednisolone (corticosteroid): 5.5% (w/v);
ramipril (an angiotensin converting enzyme inhibitor): 2% (w/v);
timolol (beta-blocking agent): 0.5% (w/v).

Unless otherwise indicated, administration of theses drugs was performed as follows:
each patient received topically, in one eye, one drop of each of the indicated drug(s) (apraclonidine, bevacizumab, brimonidine, dexamethasone, indomethacin, neosynephrine, prednisolone, ramipril and/or timolol); then,
every hour, over a period of 7 hours, the conjunctivae of the two eyes were exposed to one drop of fluorescein solution;
8 hours after administration of the first drug (apraclonidine, bevacizumab (apraclonidine, bevacizumab, brimonidine, dexamethasone, indomethacin, neosynephrine, prednisolone, ramipril and/or timolol), the fundus fluorescence in the two eyes was measured and analysed.

### Example 1

The experimental technique was based on ocular fundus fluorescence: Five patients were used in this study. Each patient received topically in one eye a drop of either apraclonidine or brimonidine or neosynephrine or timolol. Then every hour, the conjunctivae of the two eyes were exposed every hour to a 10% fluorescein solution. 8 hours later the fundus fluorescence in the two eyes was measured and analysed.

The fluorescence in the eye which received the drug carrier (brimonidine, apraclonidine, neosynephrine, or timolol) was stronger than in the eye receiving only fluorescein indicating that this drug carrier had enhanced the delivery of 10% fluorescein to the posterior segment (chorio-retina; optic nerve head). The following is a synopsis and results of the study undertaken:

### First Case: Iopidine and Ocular Fundus

| | |
|---|---|
| Right eye: | left eye: |
| lopidine + fluorescein | fluorescein |

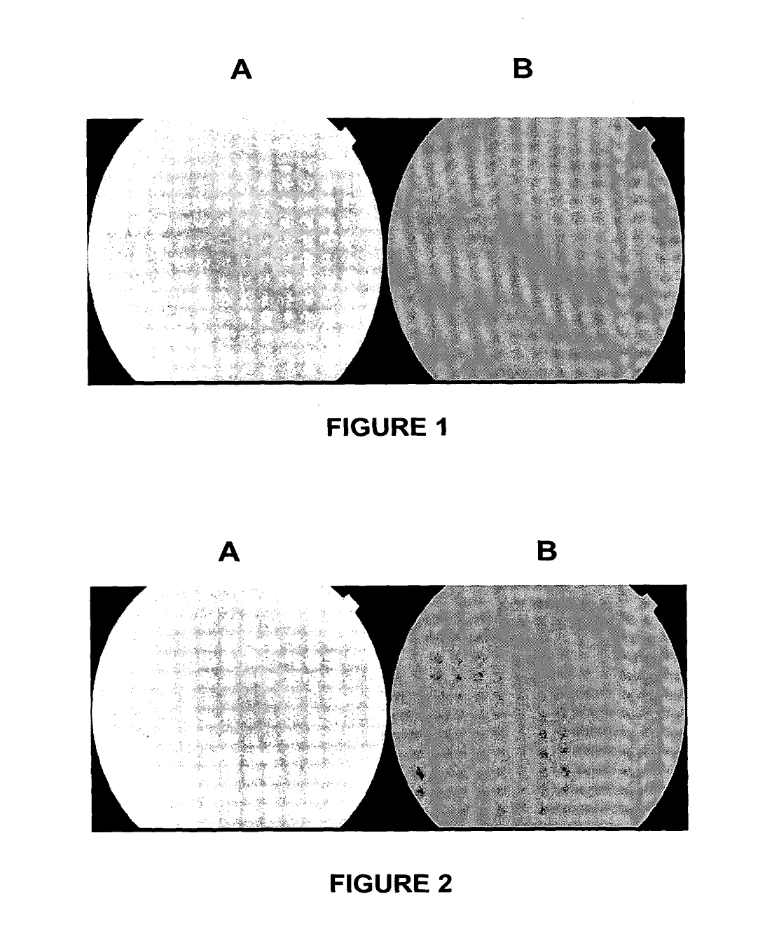

The results of the fundus fluorescence are shown in FIG. 1. FIG. 1A is the result obtained using apraclonidine and fluorescein. FIG. 1B is the result obtained using only fluorescein. These results show that the fluorescence is stronger in the eye that was administered iopidine and fluorescein.

### Second Case: Neosynephrine and Ocular Fundus

| | |
|---|---|
| Right eye: | left eye: |
| Neosynephrine + fluorescein | fluorescein |

The results of the fundus fluorescence are shown in FIG. 2. FIG. 2A is the result obtained using neosynephrine and fluorescein. FIG. 2B is the result obtained using only fluorescein. These results show that the fluorescence is stronger in the eye that was administered neosynephrine and fluorescein.

### Third Case: Timolol and Ocular Fundus and Anterior Segment

| | |
|---|---|
| Right eye: | left eye: |
| fluorescein | timolol + fluorescein |

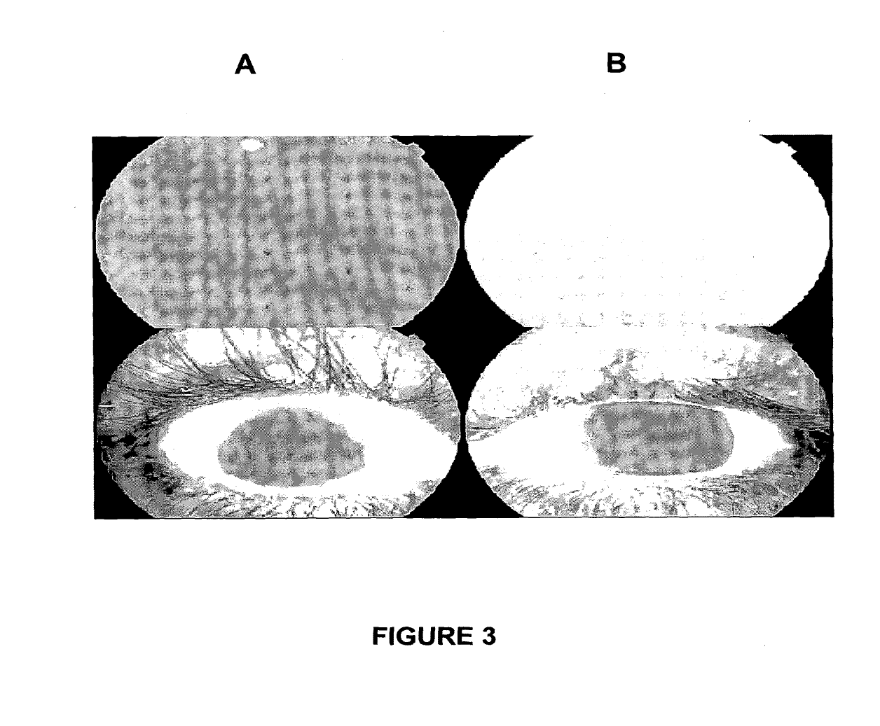

The results of the fundus fluorescence are shown in FIG. 3. FIG. 3A is the result obtained using only fluorescein. FIG. 3B is the result obtained using only timolol and fluorescein. These results show that the fluorescence is stronger in the eye that was administered timolol and fluorescein (ocular fundus). However, fluorescence is the same in both anterior segments.

### Forth Case: Apraclonidine and Ocular Fundus and Anterior Segment

| | |
|---|---|
| Right eye: | left eye: |
| fluorescein | Apraclonidine + fluorescein |

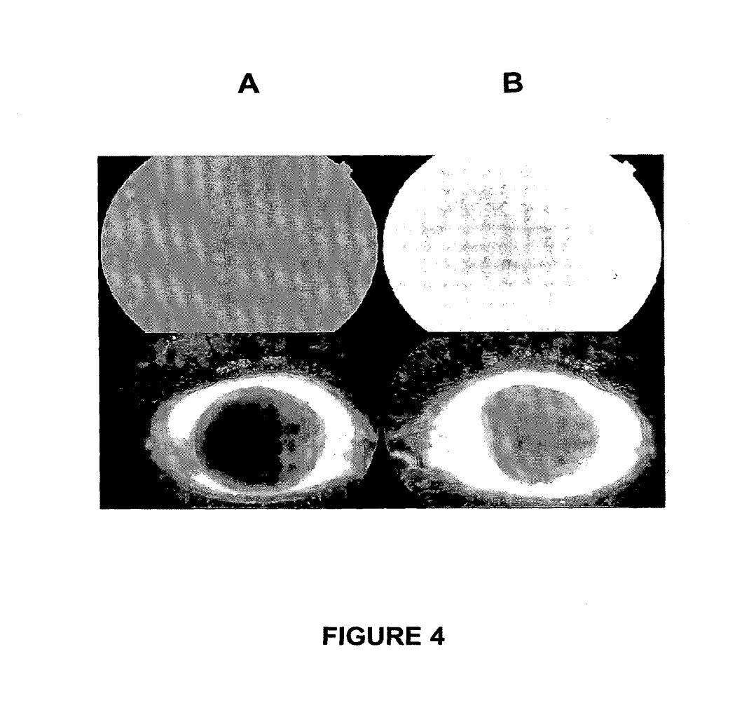

The results of the fundus fluorescence are shown in FIG. 4. FIG. 4A is the result obtained using only fluorescein. FIG. 4B is the result obtained using apraclonidine and fluorescein. These results show that the fluorescence is stronger in the eye that received apraclonidine and fluorescein (ocular fundus and anterior segment).

### Fifth Case: Brimonidine and Ocular Fundus and Anterior Segment

| | |
|---|---|
| Right eye: | left eye: |
| fluorescein | Brimonidine + fluorescein |

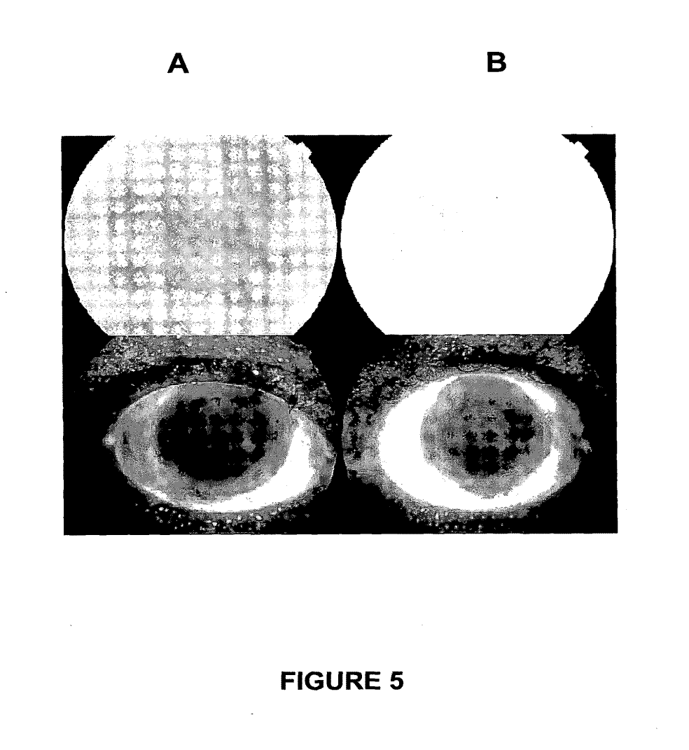

The results of the fundus fluorescence are shown in FIG. 5. FIG. 5A is the result obtained using only fluorescein. FIG. 5B is the result obtained using brimonidine and fluorescein. These results show that the fluorescence is stronger in the eye that was administered brimonidine and fluorescein (ocular fundus and anterior segment).

Collectively, these results demonstrated that the fluorescence is stronger in the eye which received the drug carrier. It works out that exposure of the conjunctival formices to a 10% fluorescein solution leads to a maximum vitreous concentration of 1.5×10⁻¹² g/ml, 7 hours later. For fluorescein this brings it into the range of therapeutic concentrations.

### Example 2

### Diabetic Retinopathy

Diabetic retinopathy is the leading cause of new blindness in individuals under 65 years of age. Diabetic retinopathy can be classified into non-proliferative diabetic retinopathy (NPDR) and proliferative diabetic retinopathy (PDR). The clinical features of NPDR include microaneurysms, intraretinal hemorrhages, hard exudates, nerve fiber layer infarcts or cotton wool exudates and intra retinal microvascular abnormalities (IRMA). The clinical picture of PDR includes the features from NPDR in addition to proliferating new vessels on the optic nerve head, retina or iris.

Diabetic macular oedema is a principal cause of visual loss in diabetic patients. Two examination techniques are very useful in evaluating diabetic retinopathy: fluorescein angiography and optical coherence tomography.

Fluorescein angiography is used to detect several of the retinal vascular abnormalities. The dye delineates structural vascular alterations, such as aneurysms or neovascularization, changes in blood flow such as ischemia and vascular occlusion are seen as an interruption of the normal perfusion pattern. Abnormal vascular permeability is seen as a leaking cloud of dye-stained oedema fluid increasing overtime.

Optical coherence tomography (O.C.T) may be more sensitive in evaluating diabetic macular oedema than slit-lamp examination. In addition, central macular thickness correlates with visual acuity even better than fluorescein leakage.

The response of macular oedema to the administration of one drug such as an anti-VEGF agent or an anti-inflammatory treatment such as topical corticosteroids, non-steroidal anti-inflammatory agents or angiotensin converting enzyme inhibitors, can be documented accurately by OCT imaging.

Diabetic patients were treated with one of the following drugs given topically: a corticosteroid (dexamethasone (Tobradex®) or prednisolone), a non steroidal anti-inflammatory agent (indomethacin (Indocollyre®)), an anti-VEGF (bevacizumab (Avastin®)) or an angiotensin converting enzyme inhibitor (Ramipril). These drugs were given in combination with a drug carrier: an alpha adrenergic agonist agent or a beta-blocking agent or alpha adrenergic agonist agent combined with a beta-blocker, for enhancing the delivery of the drug to the retina.

The following study was undertaken on six patients having non-proliferative diabetic retinopathy or proliferative diabetic retinopathy.

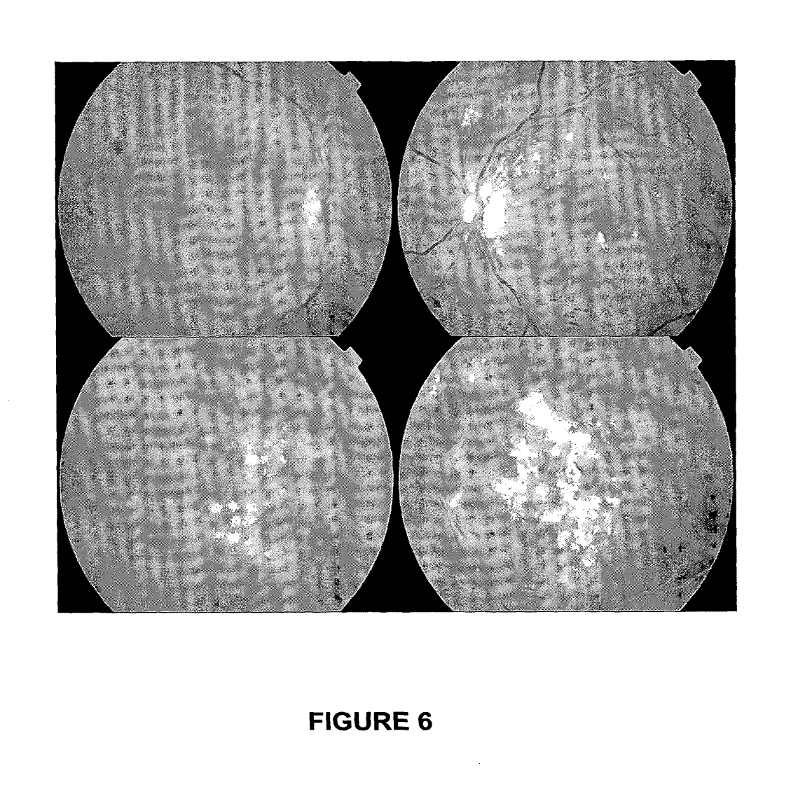

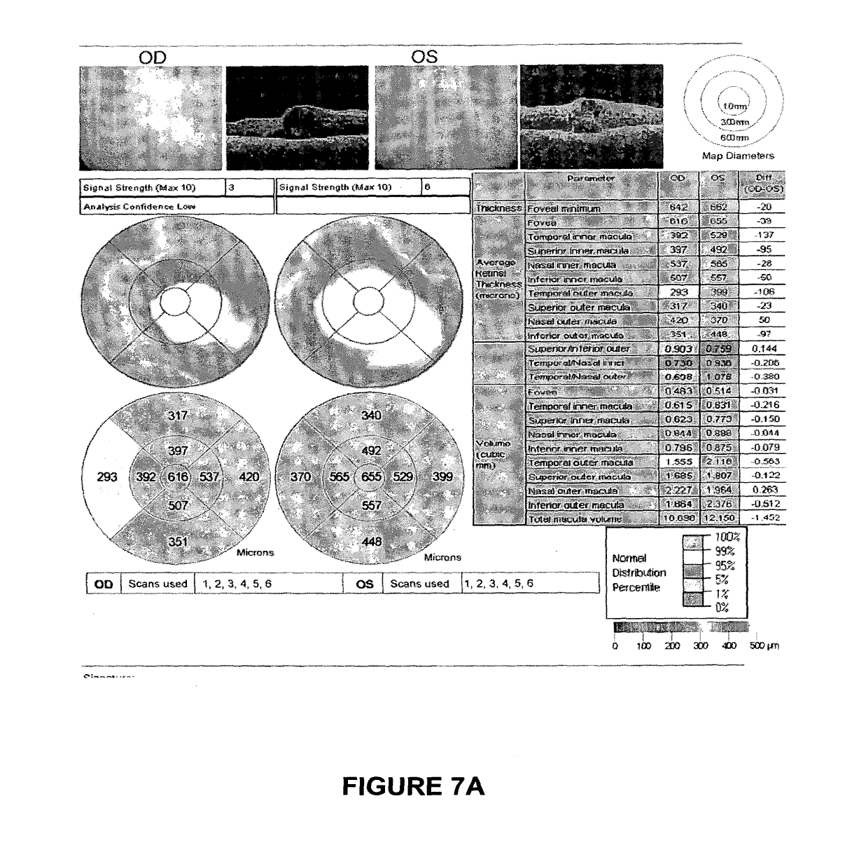

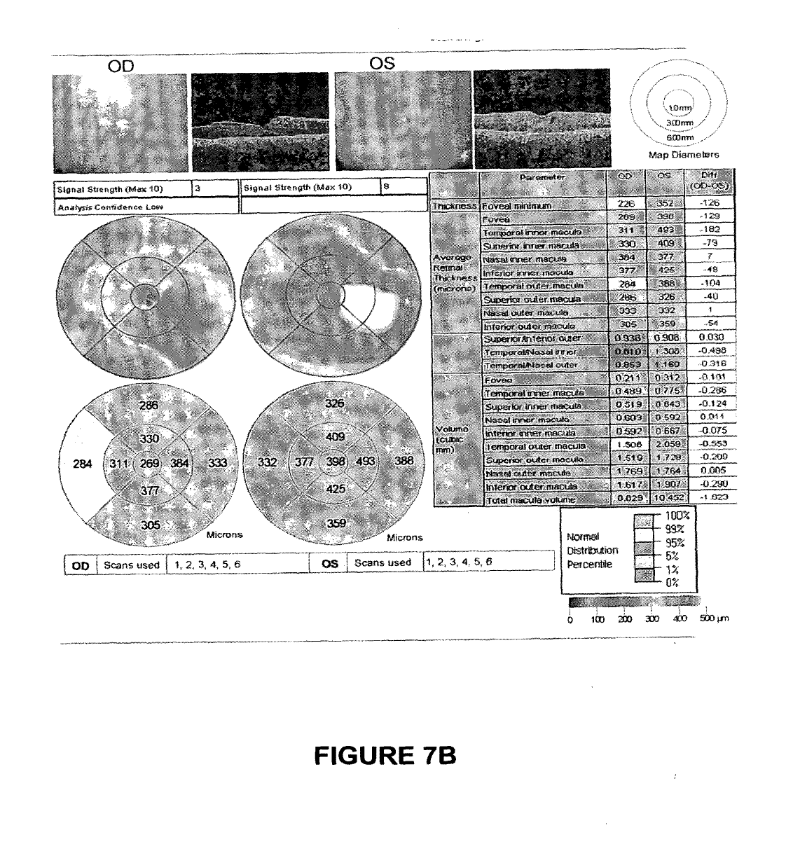

B.C.N is a diabetic patient who presented with a diabetic retinopathy (FIG. 6: ocular fundus and fluoro-angiography; FIG. 7A: OCT). He received topically a combination of apraclonidine+bevacizumab (Avastin®). Two months after treatment, a follow up OCT scan (FIG. 7B) showed that the total macula volume had decreased to 285 microns in the right eye and 402 microns in the left eye explaining visual improvement.

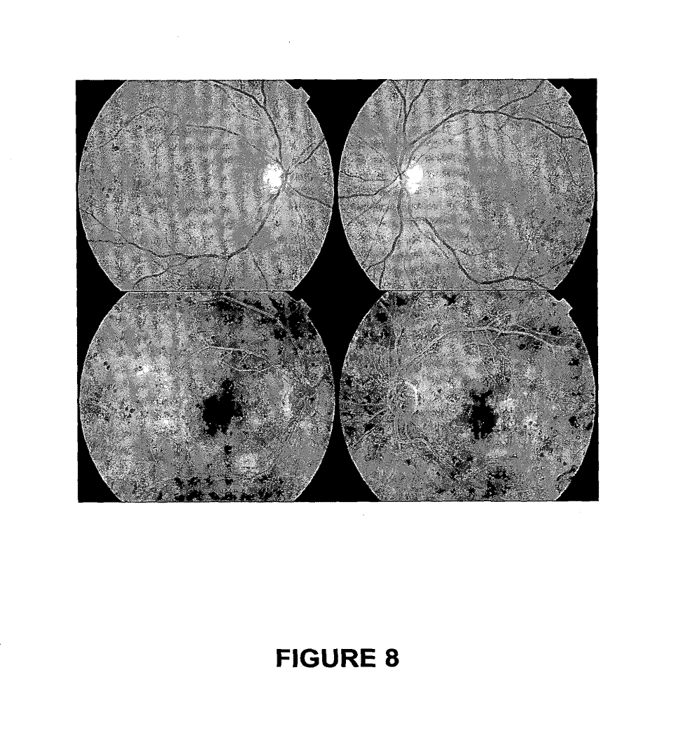

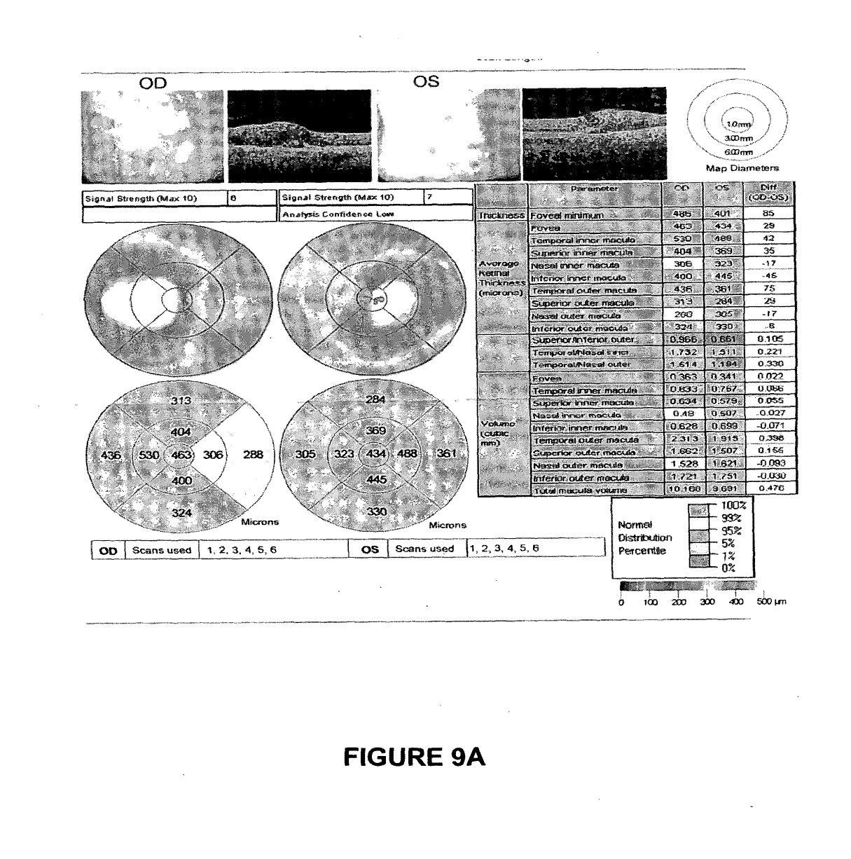

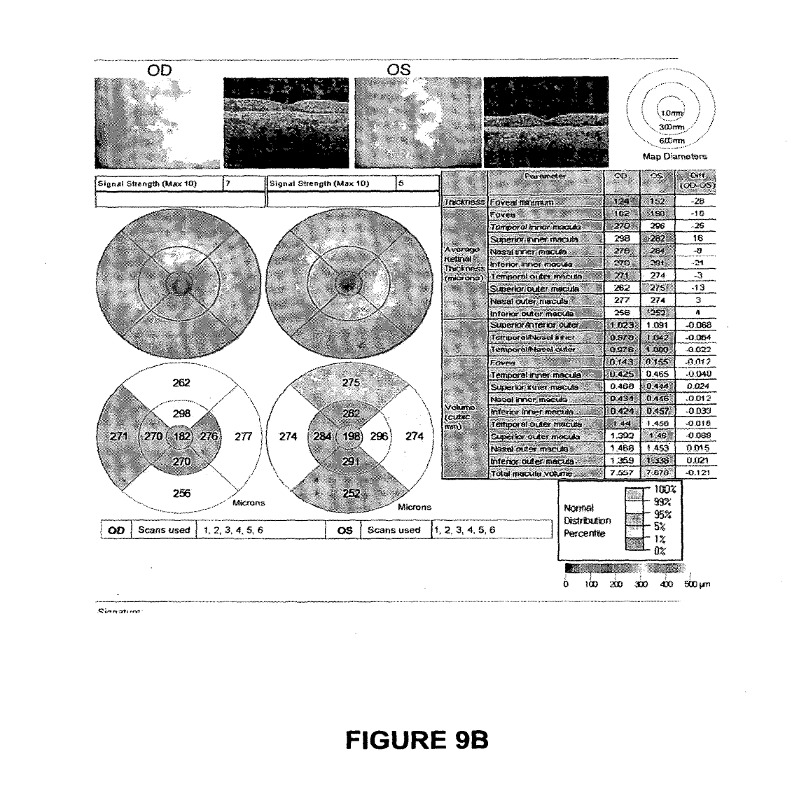

Patient G.N presented with a non proliferative diabetic retinopathy (FIG. 8: ocular fundus and fluoro-angiography; FIG. 9A: OCT). She received topically: corticosteroid+non-steroidal anti-inflammatory agent+apraclonidine. Three months later, her visual acuity was improved. A follow up OCT scan three months later (FIG. 9B) showed complete resolution of the intra-retinal and subretina fluid.

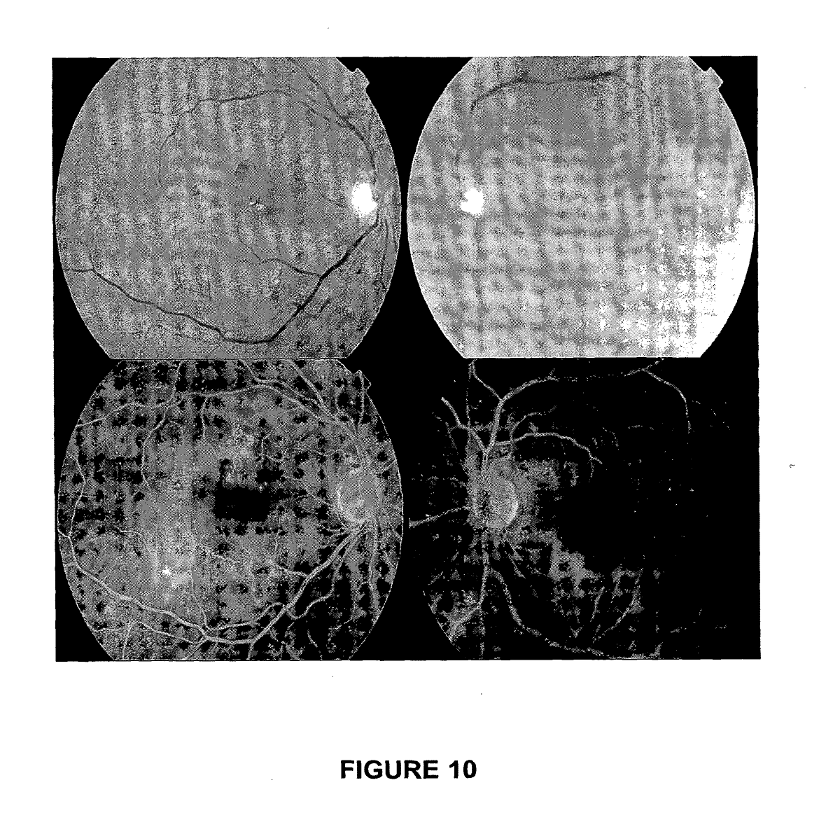

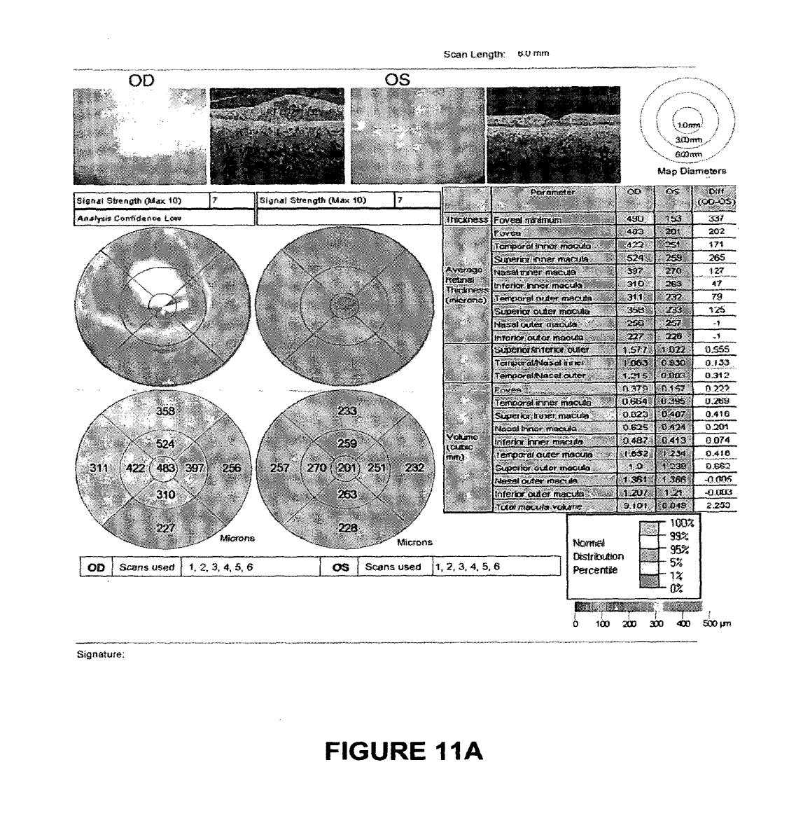

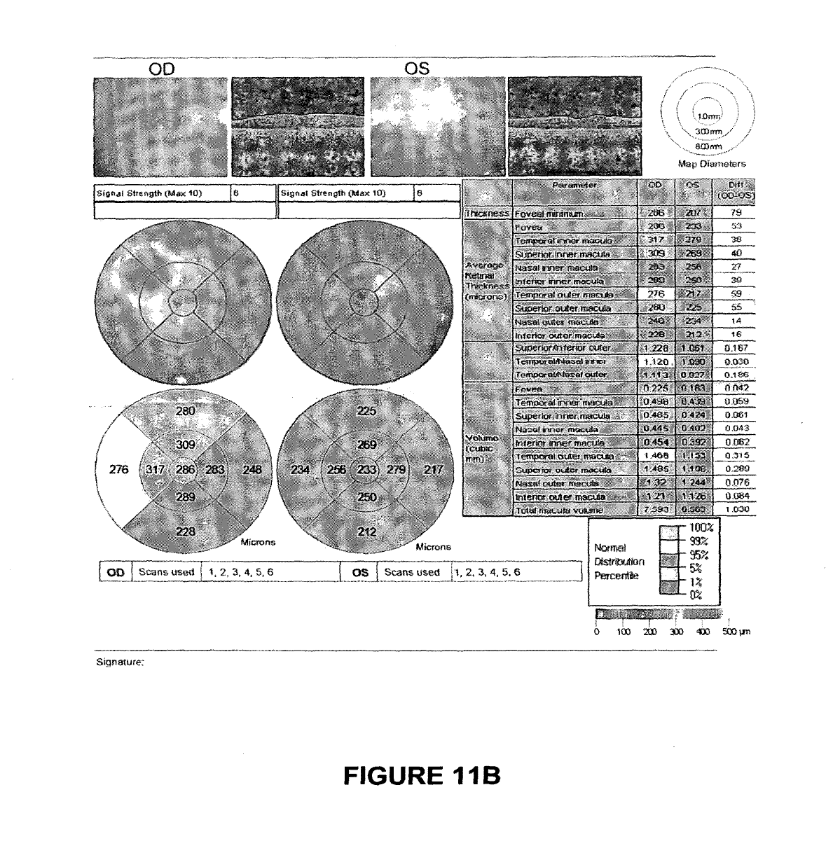

Patient M.M. presented with a non proliferative diabetic retinopathy (FIG. 10: ocular fundus and fluoro-angiography; FIG. 11A: OCT). She received topically: corticosteroid+neosynephrine. Three months later, her visual acuity improved. A follow up OCT scan (FIG. 11B) showed an almost complete resolution of the intra-retinal fluid.

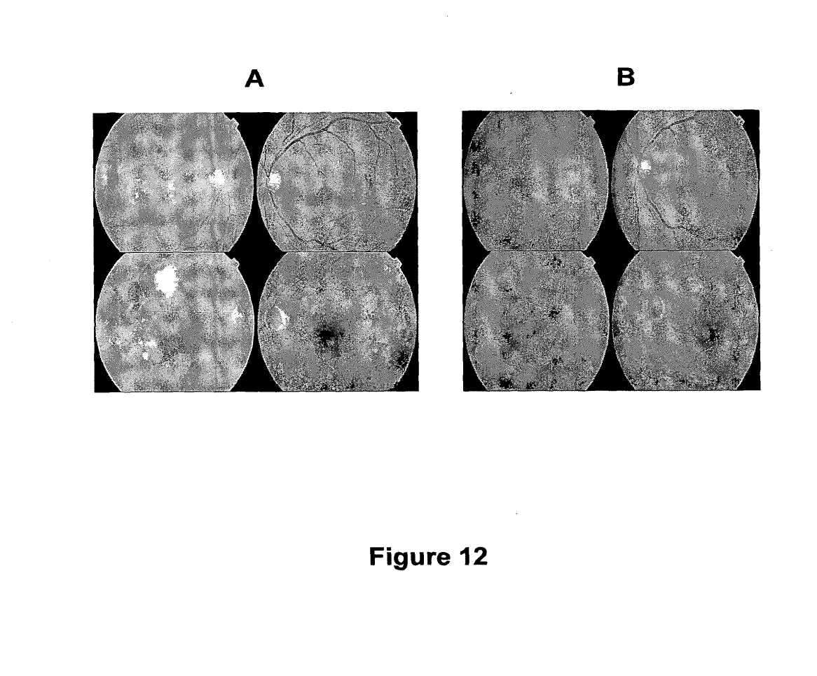

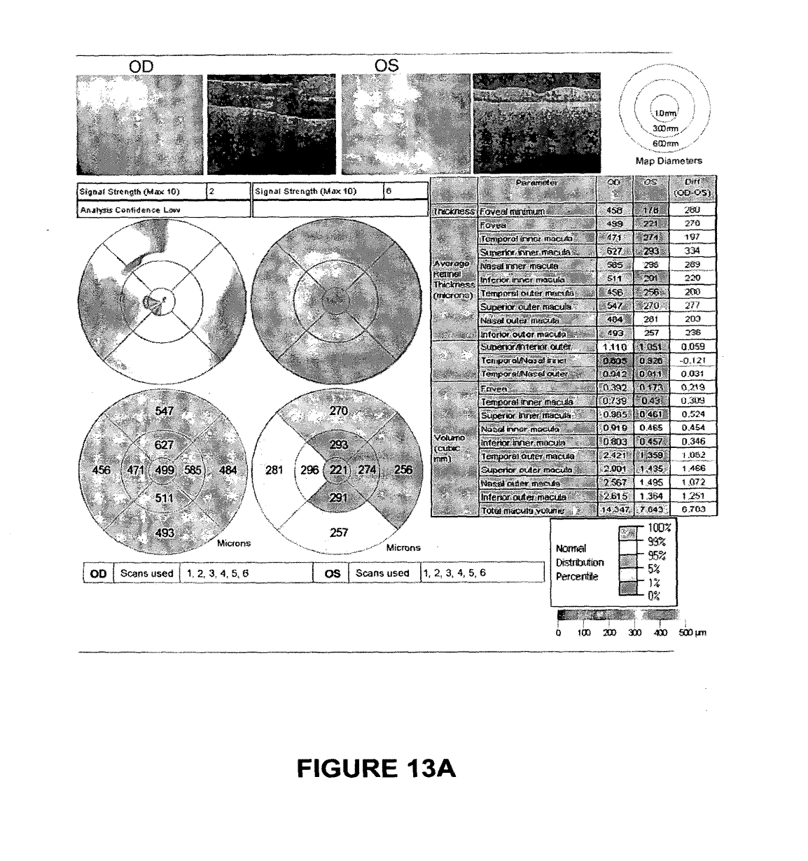

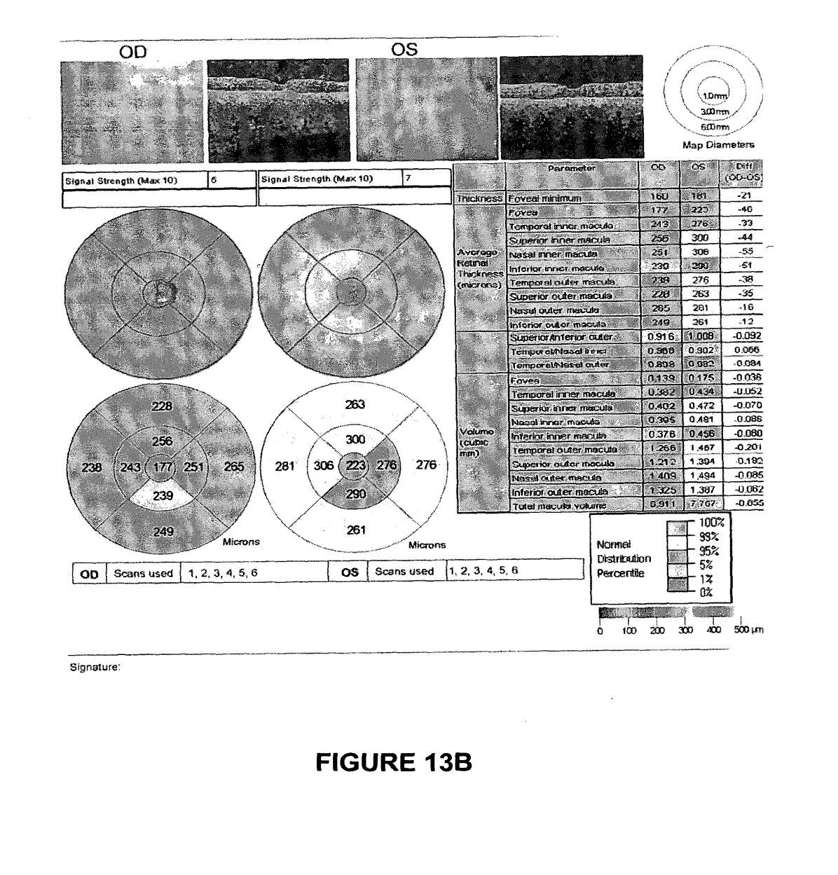

Patient H.M. presented with a proliferative diabetic retinopathy. He received a combination of brimonidine+a non-steroidal anti-inflammatory agent. An ocular fundus and fluoro-angiography were performed prior to treatment and the results are shown in FIG. 12A. Prior to the treatment and three months after the treatment, an OCT scan was taken, which is shown in FIGS. 13A and 13B respectively. Visual acuity improved after treatment. A follow up fluoro-angiography (FIG. 12B) showed the regression of new vessels and macular oedema: the retinal map analysis revealed foveal normalization.

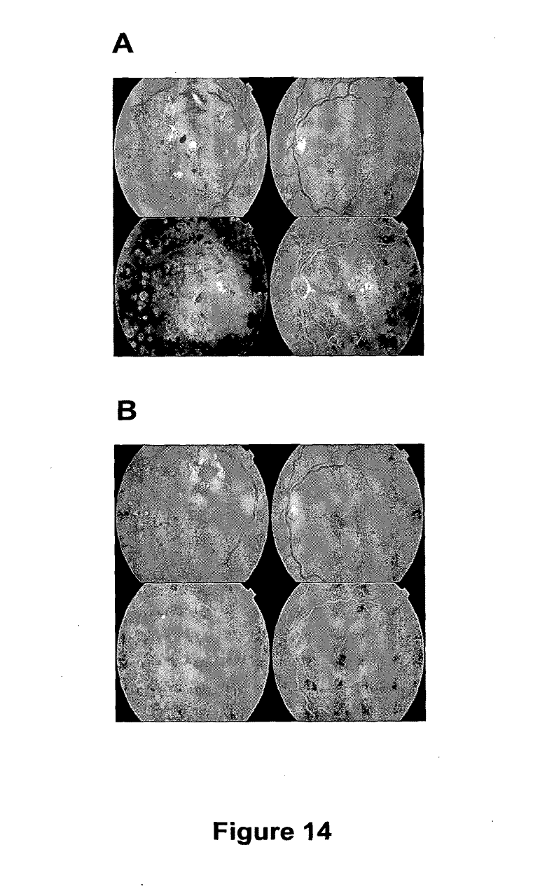

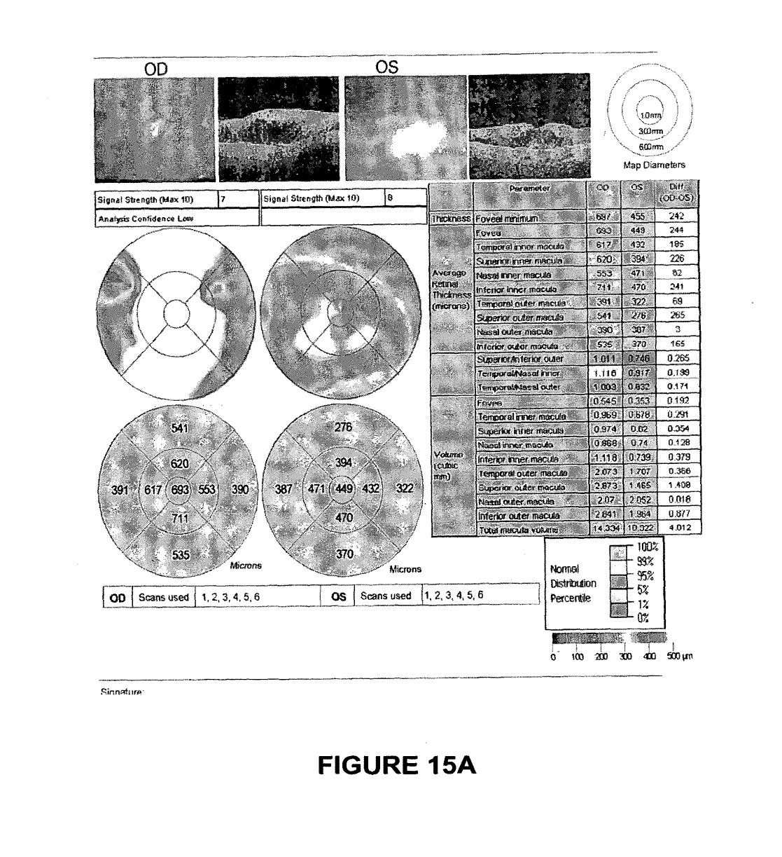

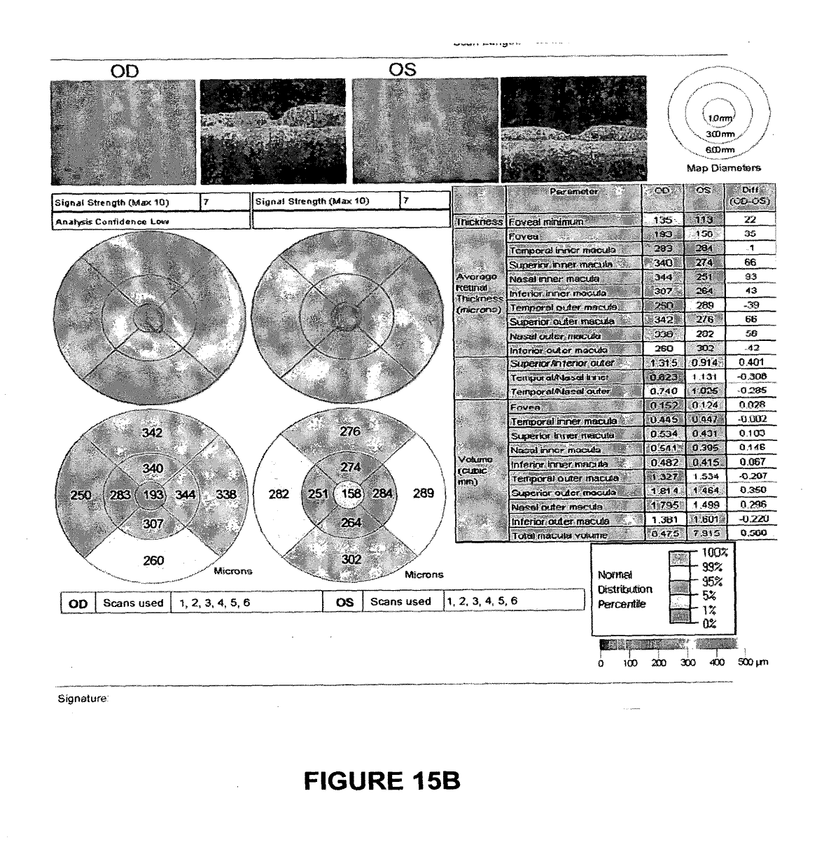

Patient N.B presented with a non proliferative diabetic retinopathy. She received topically: corticosteroid+non-steroidal anti-inflammatory agent+brimonidine. An ocular fundus and fluoro-angiography were performed prior to treatment and the results are shown in FIG. 14A. Prior to receiving the treatment an OCT scan was also taken, which is shown in FIG. 15A. Six months later her visual acuity improved. A follow up fluoro-angiography (FIG. 14B) and OCT (FIG. 15B) demonstrated a reduction of macular oedema and foveal thickening.

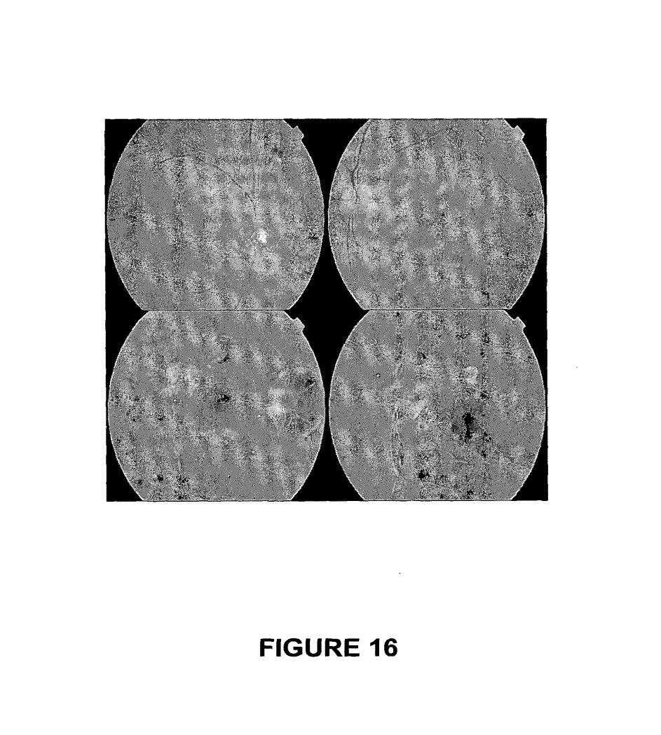

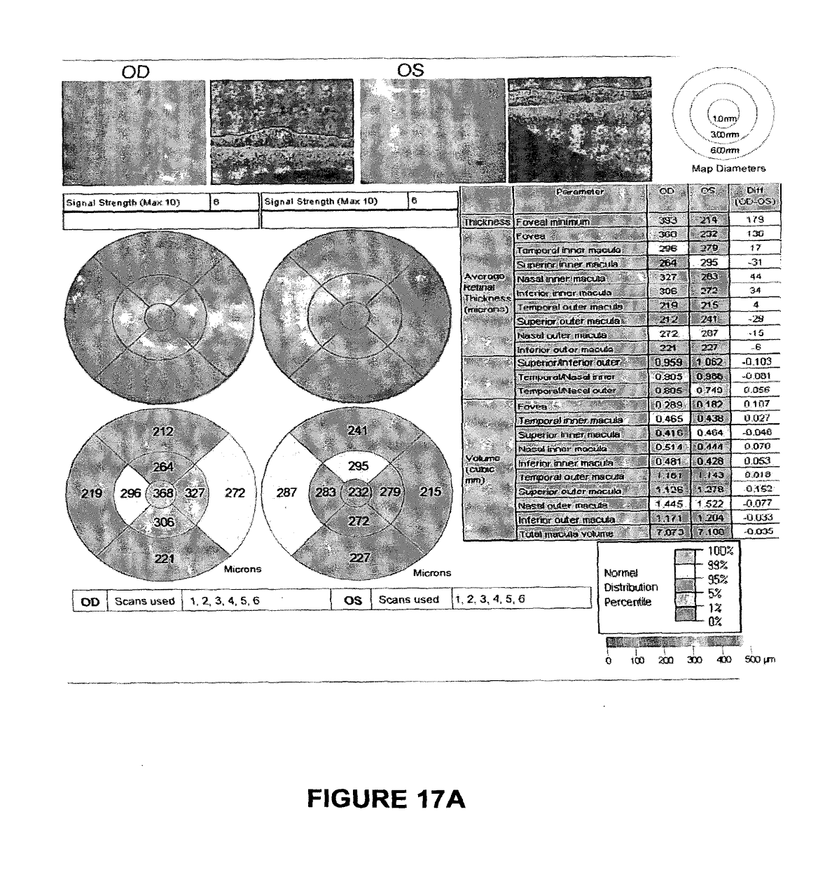

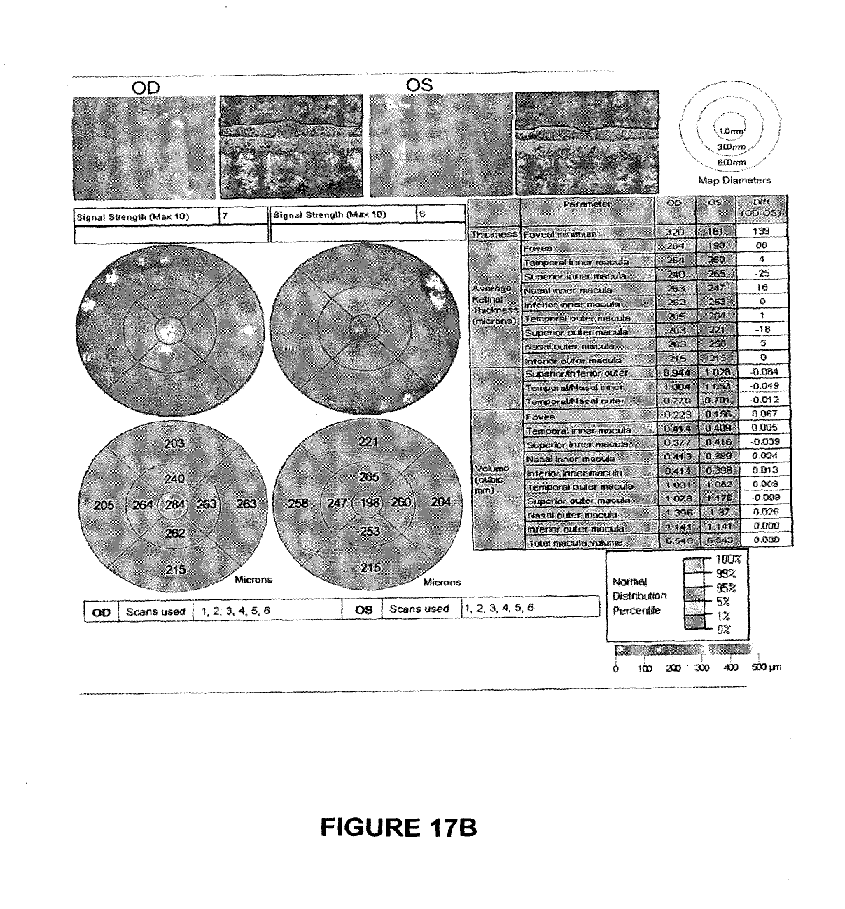

Patient M.R. presented with a diabetic retinopathy. He received topically: apraclonidine+Avastin® (anti-VGEF). Prior to receiving the treatment an OCT scan was taken, which is shown in FIG. 17A. An ocular fundus and fluoro-angiography were performed prior to treatment and the results are shown in FIG. 16. Three months later, his visual acuity improved. A follow up OCT (FIG. 17B) demonstrated and a reduction of macular oedema and foveal thickening.

As shown from the results above, the patients improved their vision with a decrease of macular oedema and neovascularization after 2 months of treatment. A follow-up OCT scan showed that the foveal thickness had decreased. An almost complete resolution of macular oedema had also been observed.

### Example 3

### Retinal Vein Occlusion

Central retinal vein occlusion (CRVO) is a common retinal vascular condition usually affecting people older then 50 years. Patients typically experience visual loss and present with dilated tortuous retinal veins and scattered intra-retinal hemorrhage in all four quadrants, cotton wool spots, optic disc swelling, and macular oedema can occur. Intra veinous fluorescein angiography shows areas of blocked fluorescence from the intra-retinal blood, staining of the vessel walls, a delayed arteriovenous phase, and nonperfused areas, and perifoveal leakage.

OCT detects macular oedema. Recent studies have shown the efficacy of intravitreal triancinolone (Aristocert®) injection in macular oedema secondary to CRVO. An anti-VEGF agent (Avastin®) when injected into the eye improved this condition.

12 patients, instead of injection, received topically either a corticosteroid (dexamethasone (Tobradex®) or prednisolone) or an anti-VEGF agent (Avastin®), associated with a delivery drug enhancer such as an alpha adrenergic agonist agent alone or combined with a beta-blocking agent.

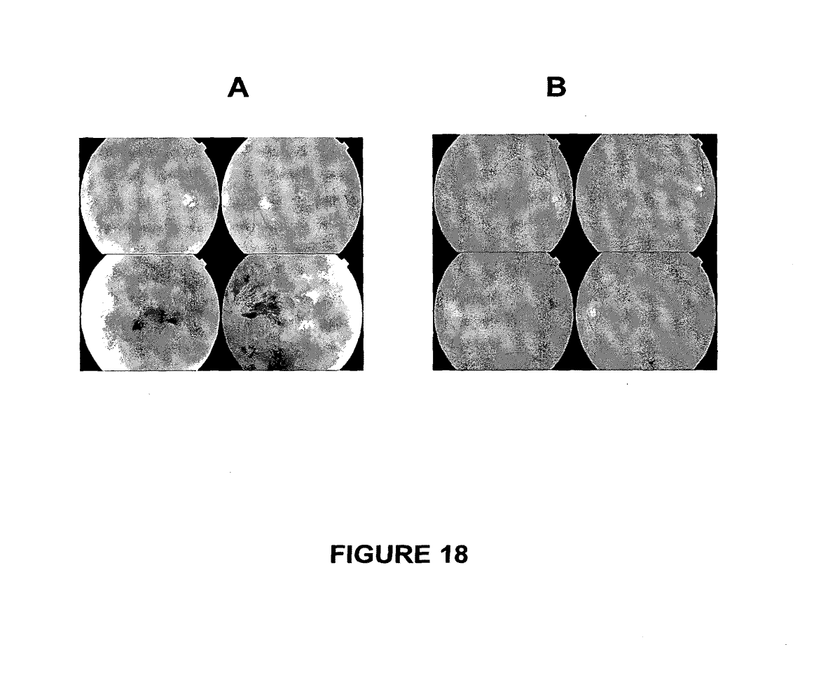

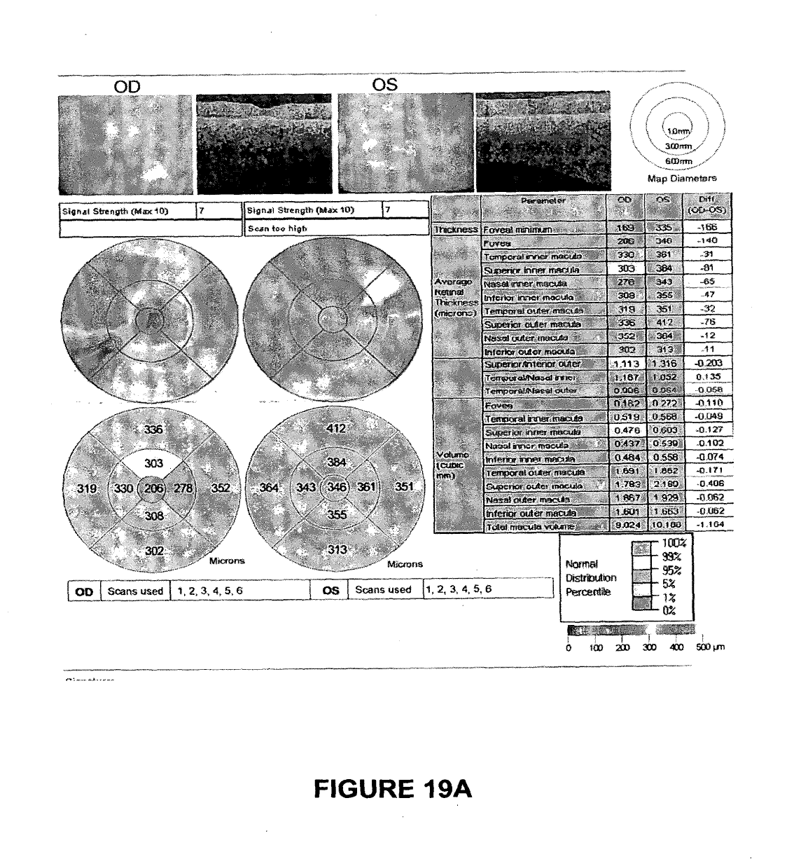

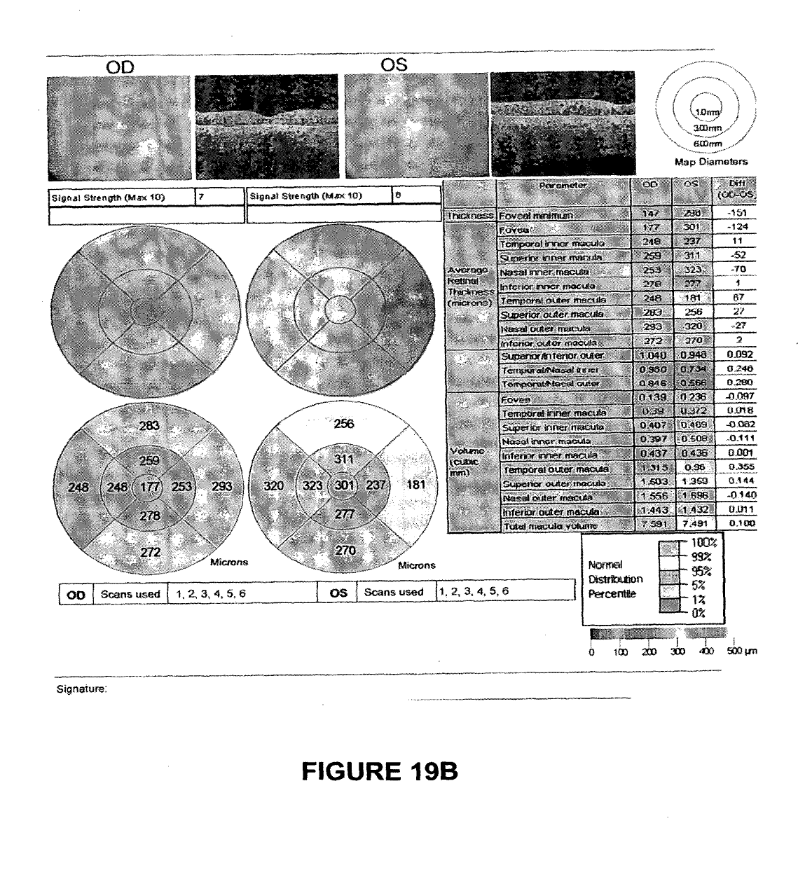

Patient N.B. presented with non proliferative diabetic retinopathy; branch vein occlusion in the left eye. She received topically: corticosteroid+non-steroidal anti-inflammatory agent+neosynephrine. Prior to receiving the treatment, an OCT scan was taken, which is shown in FIG. 19A. An ocular fundus and fluoro-angiography were performed prior to treatment and the results are shown in FIG. 18A. Three months later her visual acuity improved. A follow up fluoro-angiography (FIG. 18B) and OCT (FIG. 19B) demonstrated a reduction of macular oedema and foveal thickening.

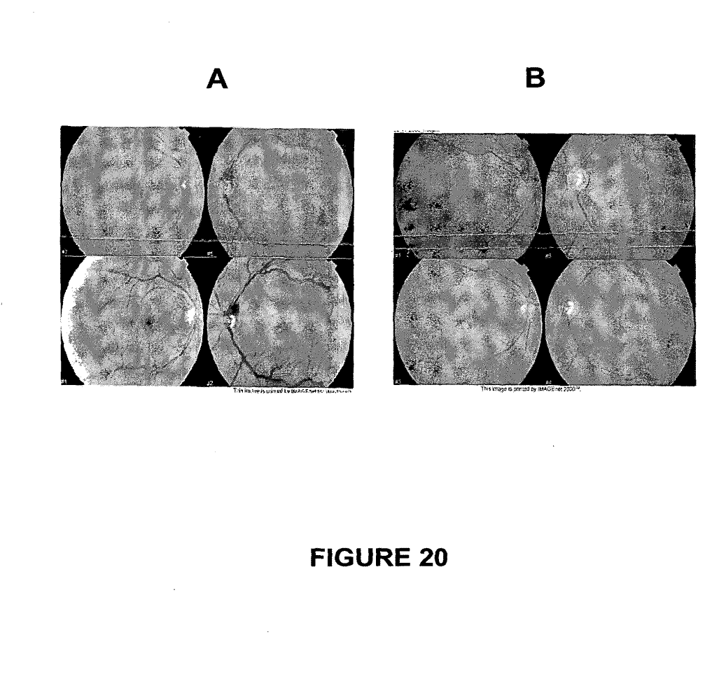

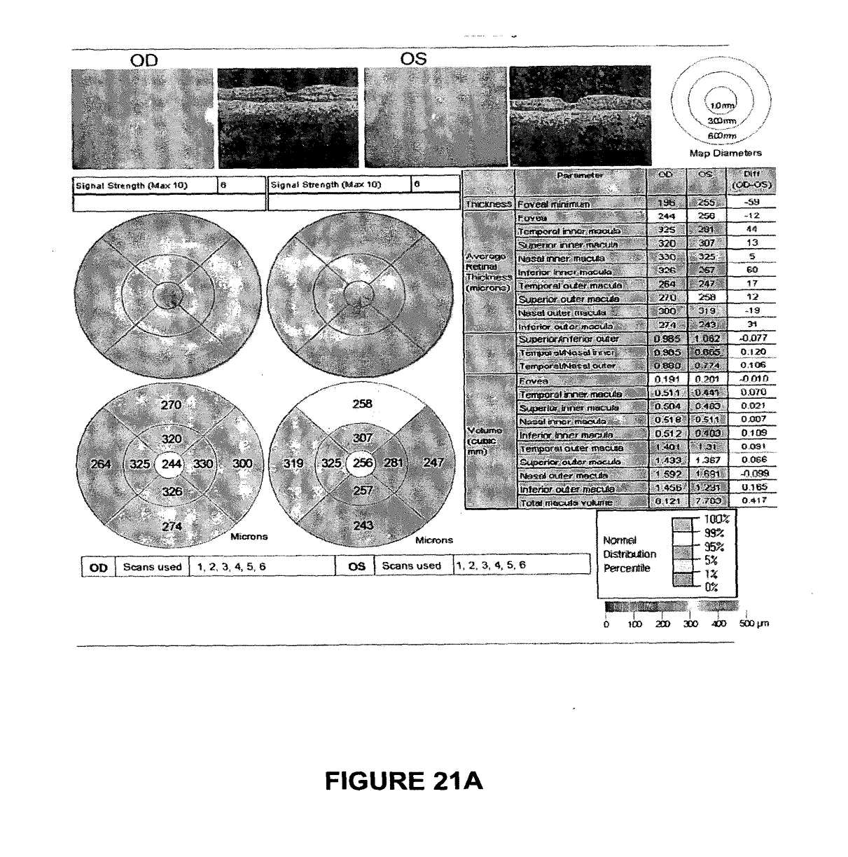

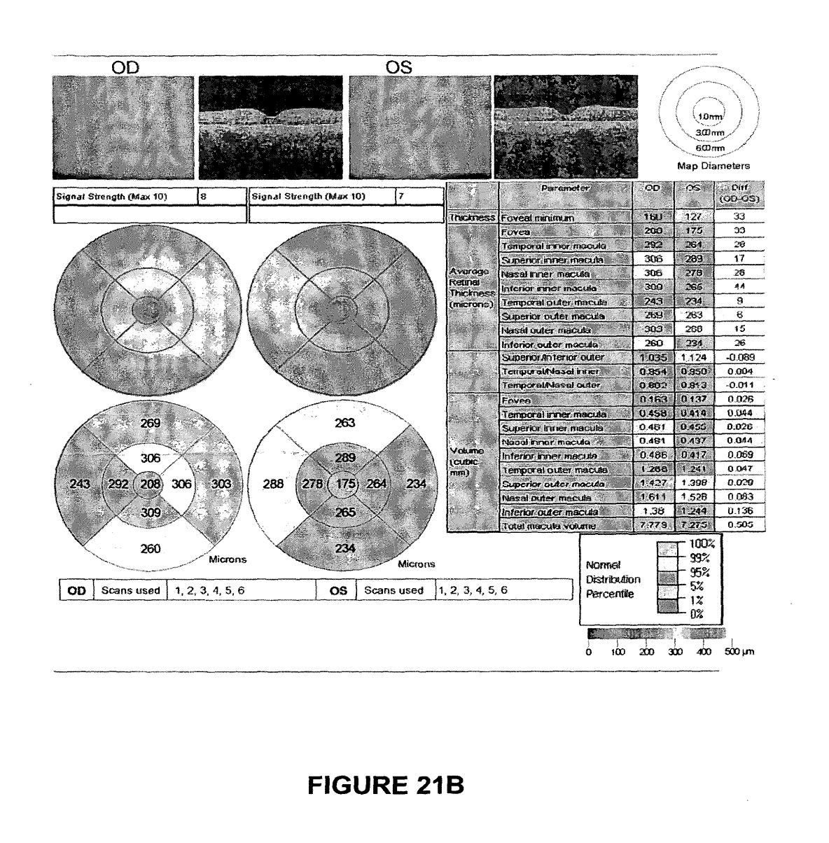

Patient B.S.T presented with imminent central retinal vein occlusion in the left eye. He received topically a non-steroidal anti-inflammatory agent+apraclonidine, Prior to receiving the treatment an OCT scan was taken, which is shown in FIG. 21A. An ocular fundus and fluoro-angiography were performed prior to treatment and the results are shown in FIG. 20A. Two weeks later his visual acuity improved. A follow up ocular fundus exam (FIG. 20B) and OCT (FIG. 21B) demonstrated the normalization of the left eye.

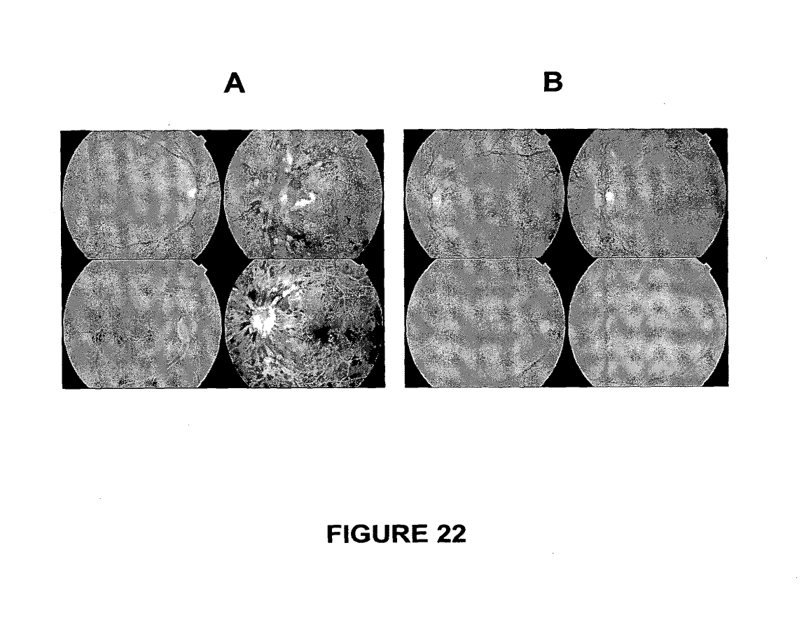

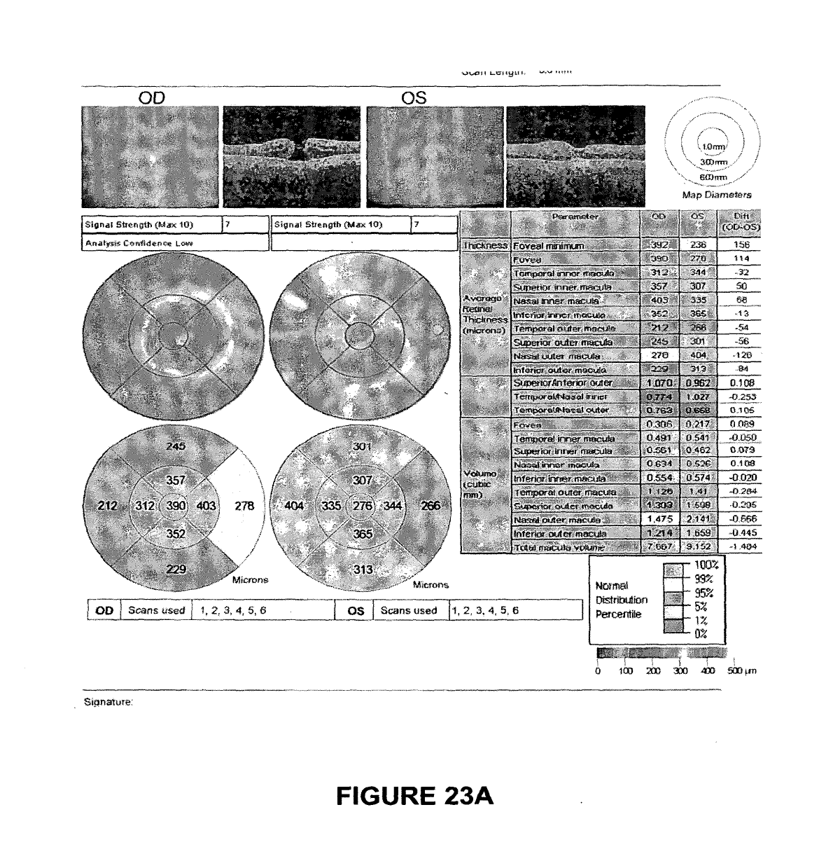

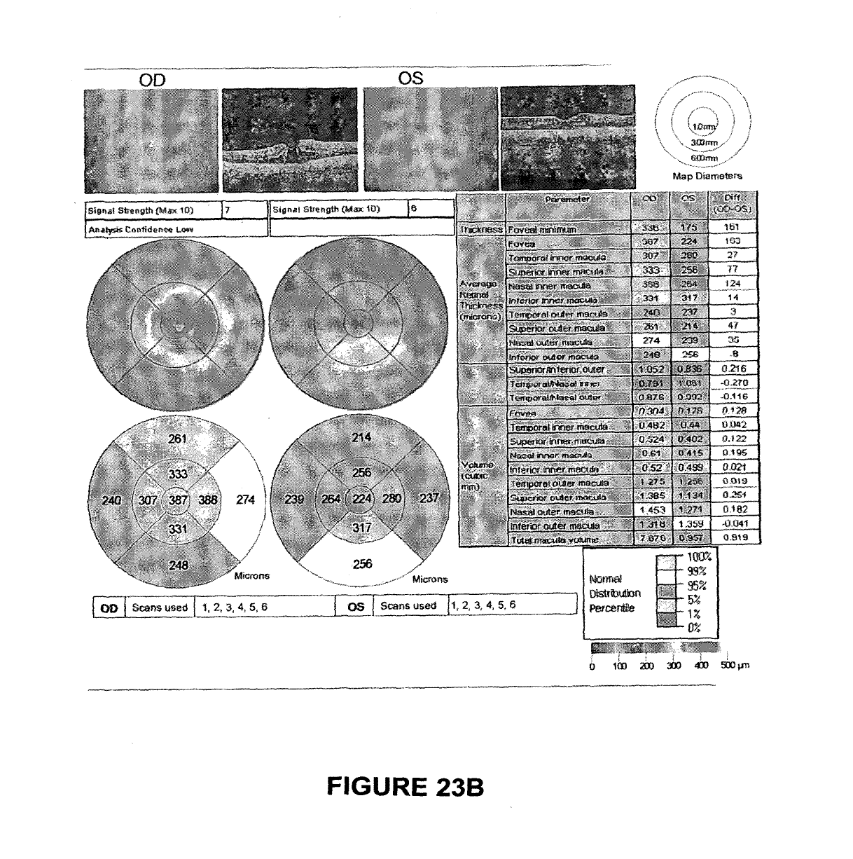

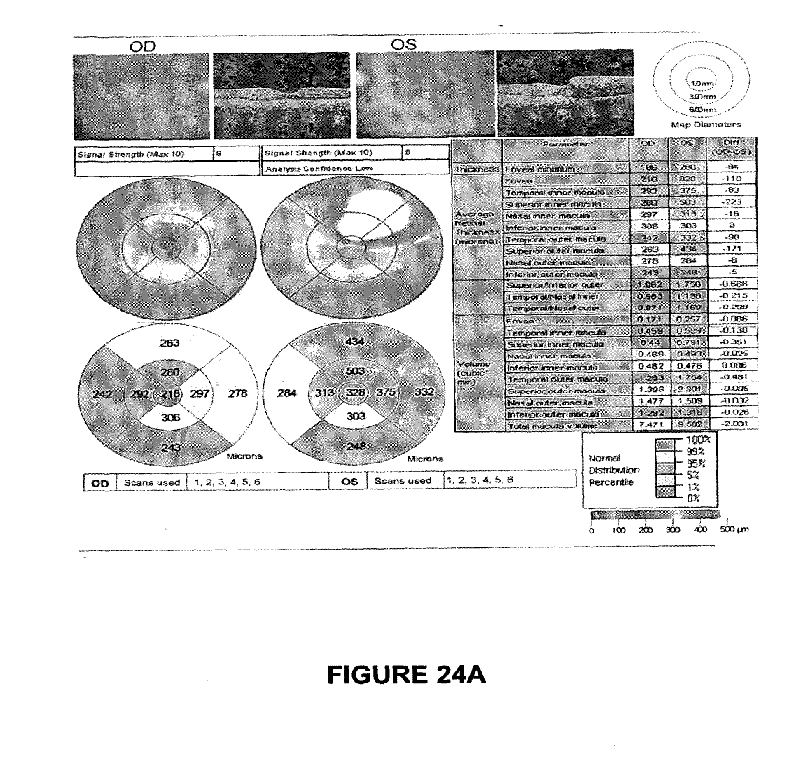

Patient A.H presented with a central retinal vein occlusion in the left eye. He received topically: non-steroidal anti-inflammatory agent+corticosteroid+neosynephrine. Prior to receiving the treatment an OCT scan was taken, which is shown in FIG. 23A. An ocular fundus and fluoro-angiography were performed prior to treatment and the results are shown in FIG. 22A. Three months later, his visual acuity increased. A follow up ocular fundus exam (FIG. 22B) revealed a fundus normalization and OCT scan (FIG. 23B) demonstrated reduction in foveal thickening.

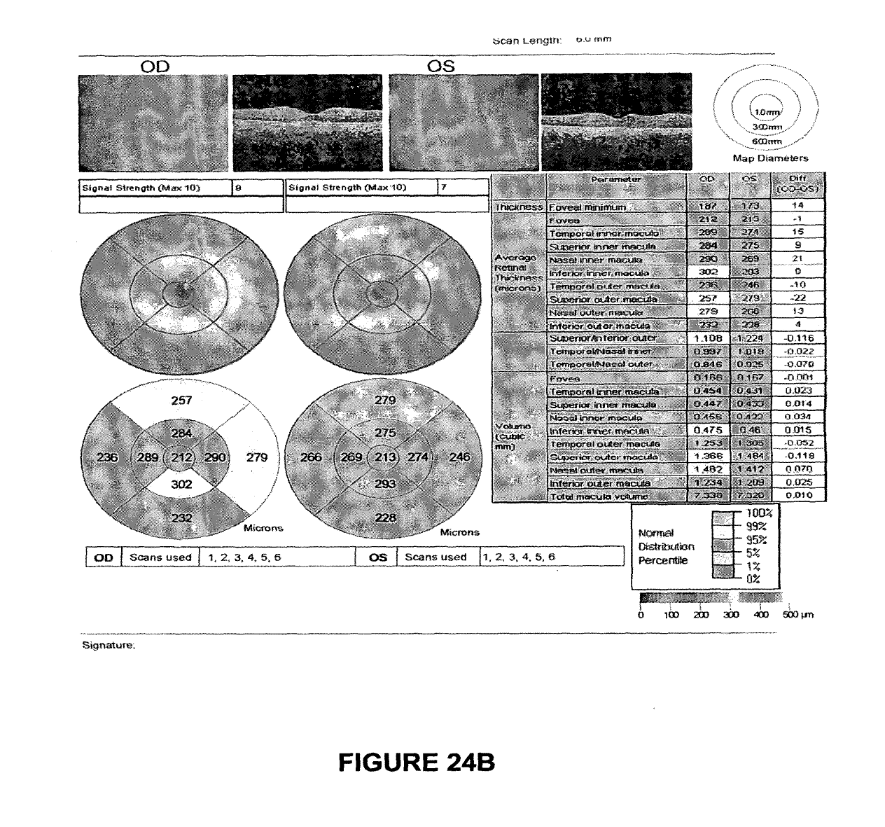

Patient S.Z presented with a branch retinal occlusion in the left eye. He received topically: Angiotensin converting enzyme inhibitor (ramipril)+apracronidine. Prior to receiving the treatment, an OCT scan was taken, which is shown in FIG. 24A. Three months later his visual acuity increased. A follow up OCT scan (FIG. 24B) demonstrated regression in macular thickening.

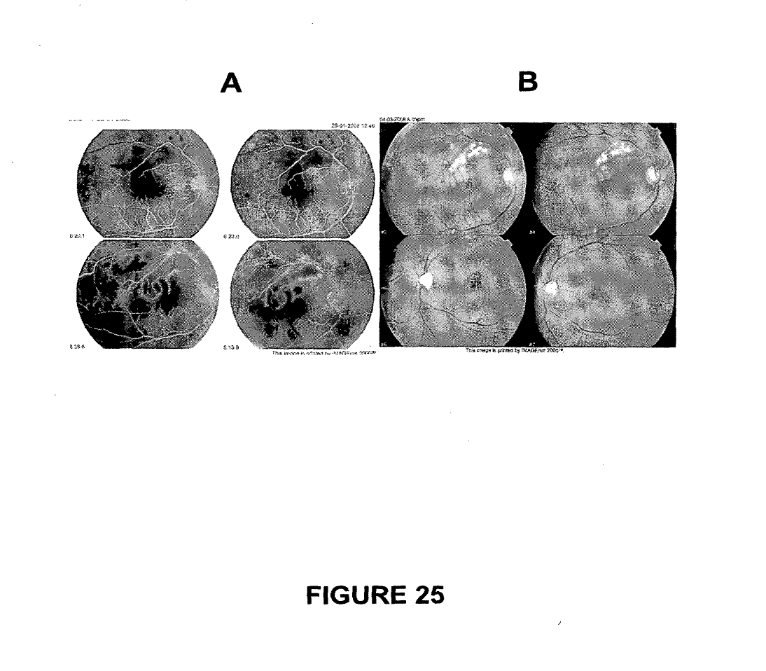

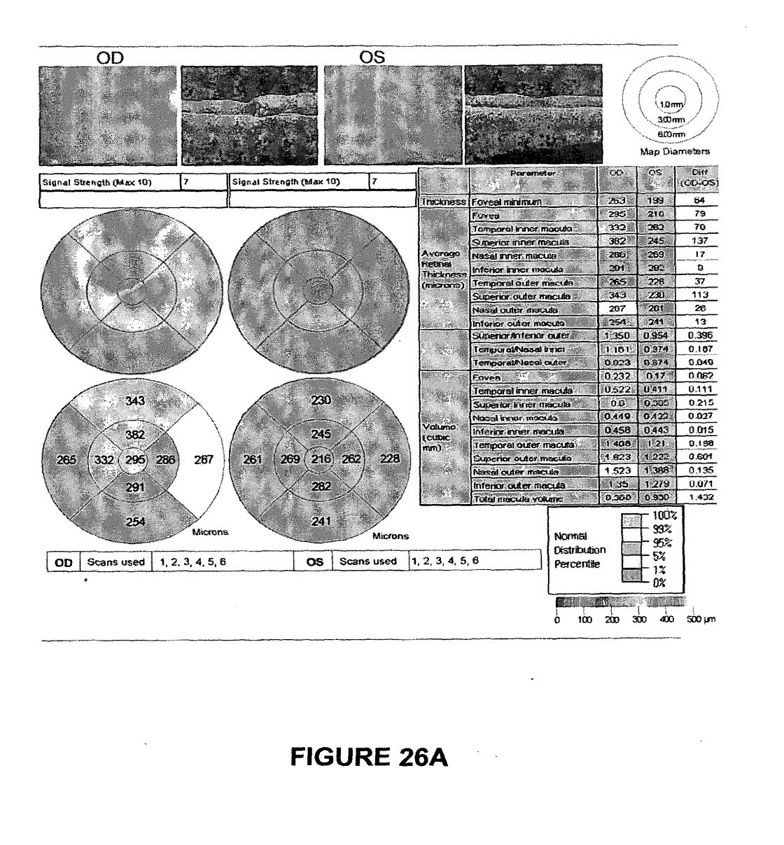

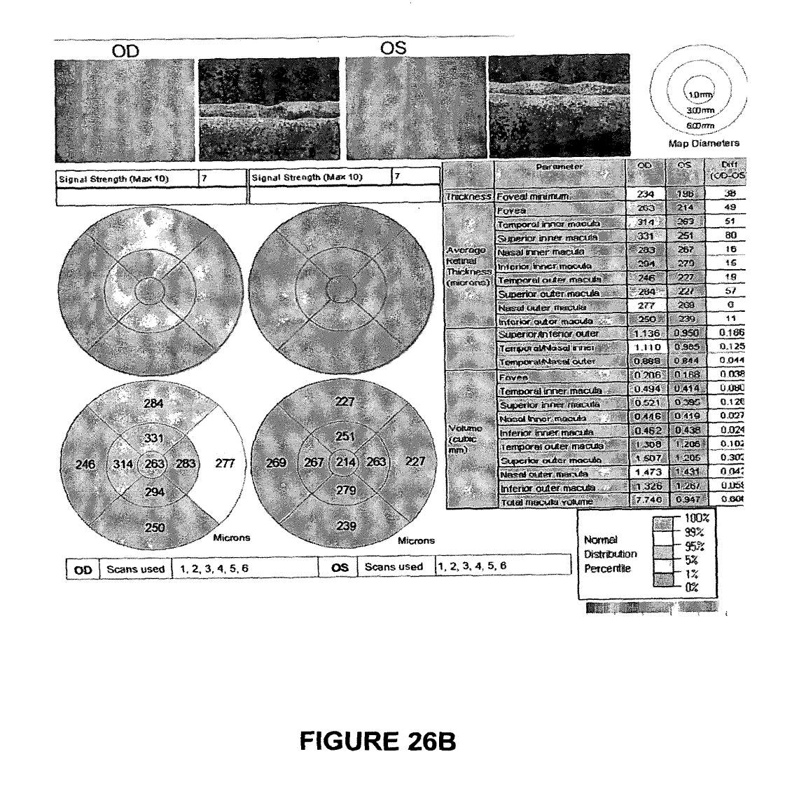

Patient D.M.H presented with a branch retinal occlusion in the right eye. He received topically: Avastin® (anti-VEGF) neosynephrine. Prior to receiving the treatment an OCT scan was taken, which is shown in FIG. 26A. An ocular fundus and fluoro-angiography were performed prior to treatment and the results are shown in FIG. 25. One month later his visual acuity increased. A follow up OCT exam revealed an improvement of the macular oedema (FIG. 26B).

As seen from the above results the patients improved their condition of visual acuity, angiographic feature and OCT results when administered the regular drug and the carrier drug.

### Example 4

### Age-Related Macular Degeneration (ARMD)

Age related macular degeneration (ARMD) is the leading cause of severe vision loss among the elderly. The cause of ARMD remains elusive and complex, with both environmental and genetic contributions. ARMD has two distinct forms known as “dry” or non-neovascular ARMD and “wet”, or neovascular ARMD. Most the severe vision loss in ARMD is caused by neovascular ARMD.

The best proven therapies for ARMD treat the neovascular form of the disease and include photocoagulation triamcinolone intraocular injection and anti-VEGF intraocular injection (Avastin®; Lucentis®; Macugen®).

The structural information provided by OCT is becoming a valuable diagnostic adjunct to fluorescein angiography. OCT is a valuable tool for probing the effects of these treatments.

Instead of an intraocular injection of an anti-VEGF agent, a corticosteroid, a non-steroidal anti-inflammatory agent and/or an angiotensin converting enzyme inhibitor, 10 patients received topically either a corticosteroid (dexamethasone (Tobradex®) or prednisolone), an anti-VEGF (bevacizumab (Avastin®)), a non steroidal anti-inflammatory agent (indomethacin (Indocollyre®)) and/or an angiotensin converting enzyme inhibitor (Ramipril), associated with a delivery drug enhancer such as an alpha adrenergic agonist agent alone or in combination with a beta-blocking agent.

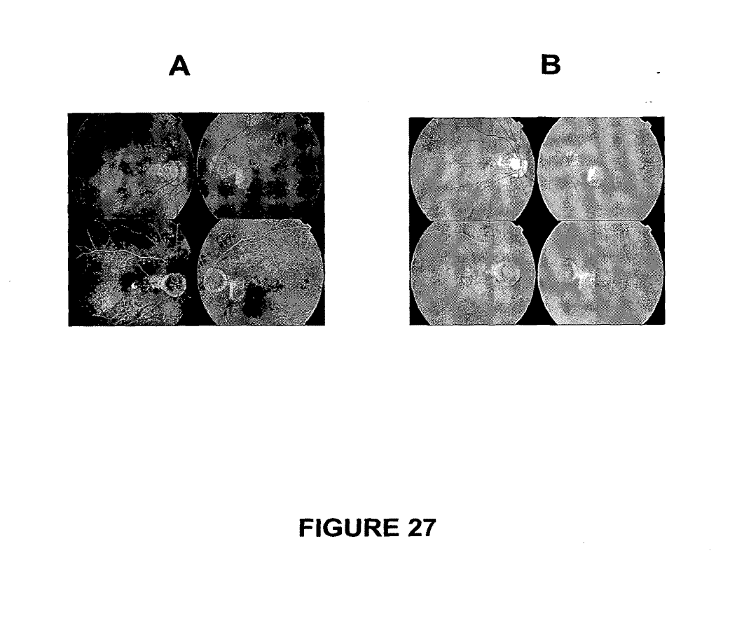

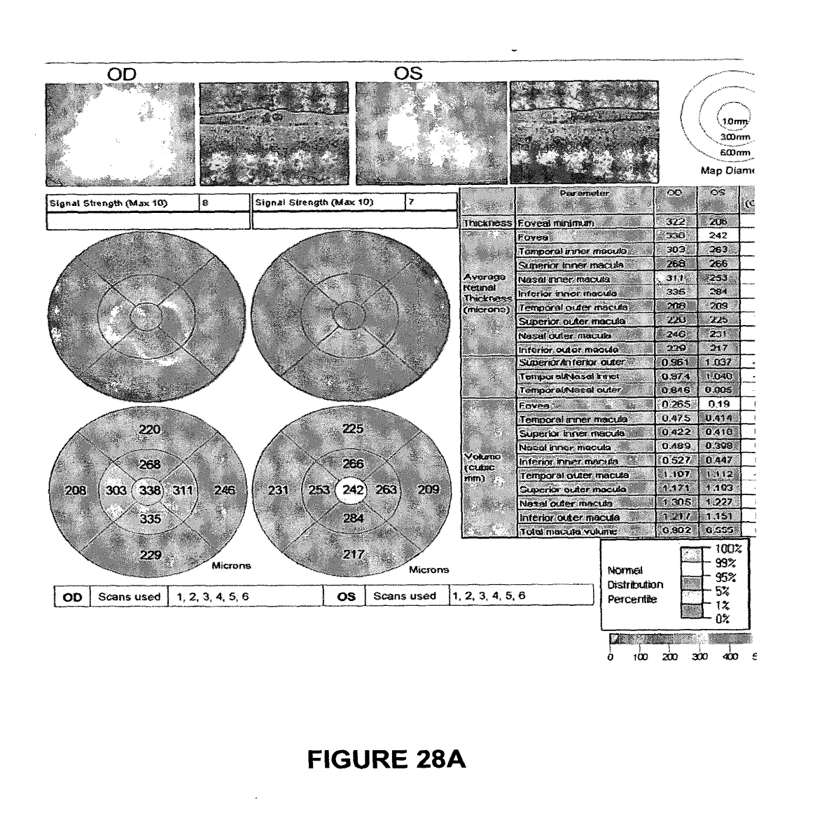

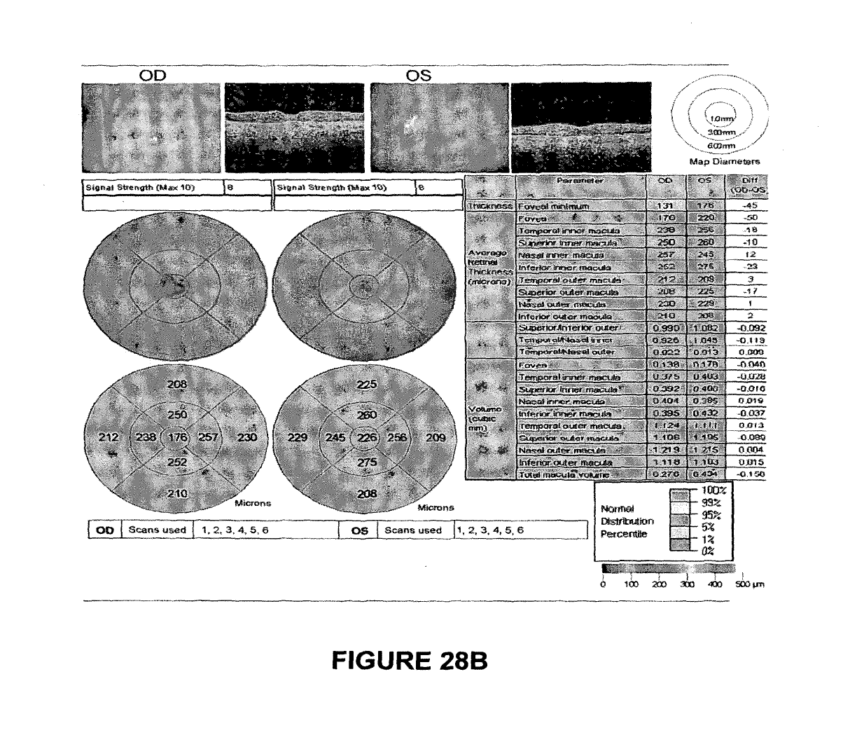

Patient CH.A presented with a two-month history of decreased vision in the right eye. An early angiographic image showed well delineated lacy subfoveal choroid new vessel (CNV) (FIG. 27A). An OCT exam showed retinal thickening with a loss of foveal contour (FIG. 28A). The patient was treated with corticosteroid+non-steroidal anti-inflammatory agent+apraclonidine. Two months after treatment, the patient's vision improved. Fluoro-angiography revealed a decreased activity of the new vessel (FIG. 27B). Follow up OCT scan (FIG. 28B) demonstrated a complete regression of macular thickening.

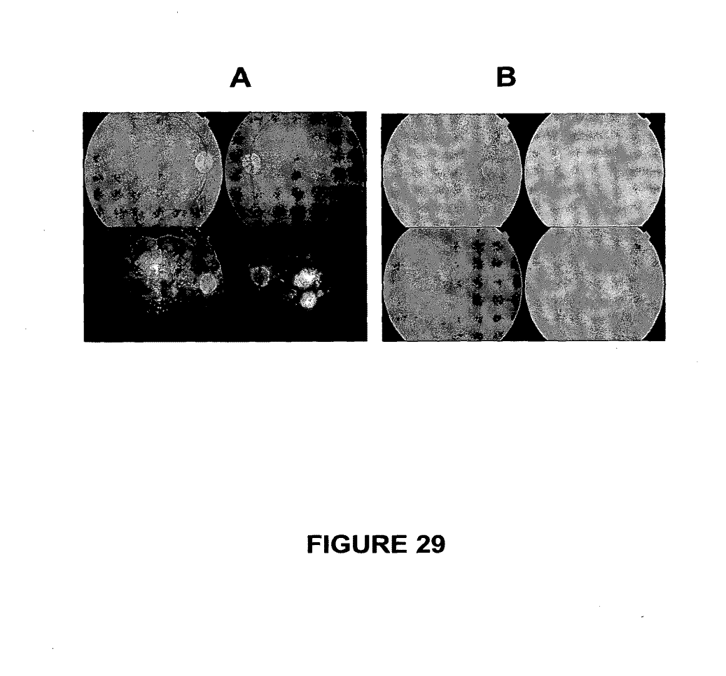

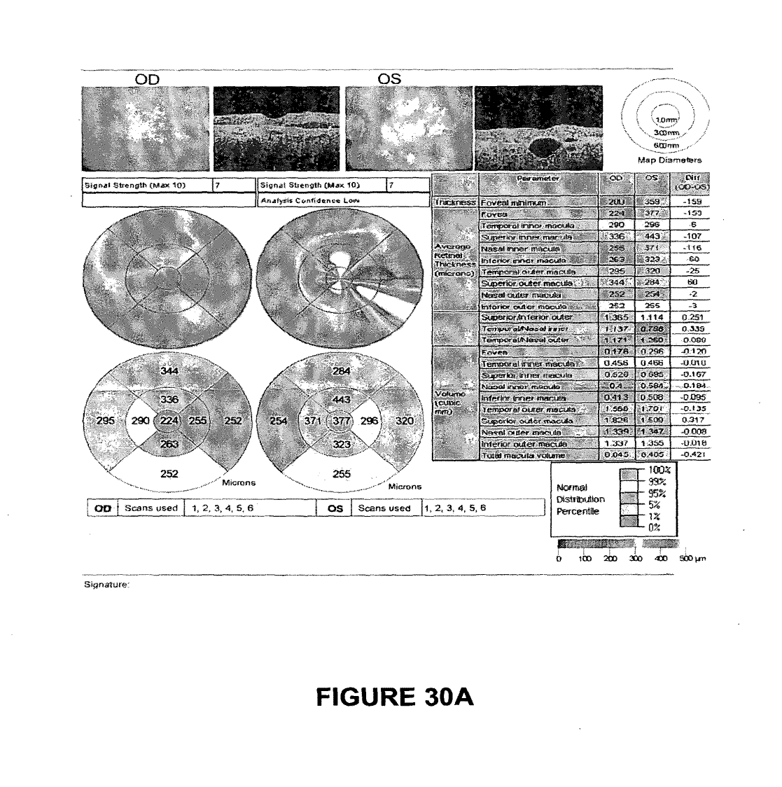

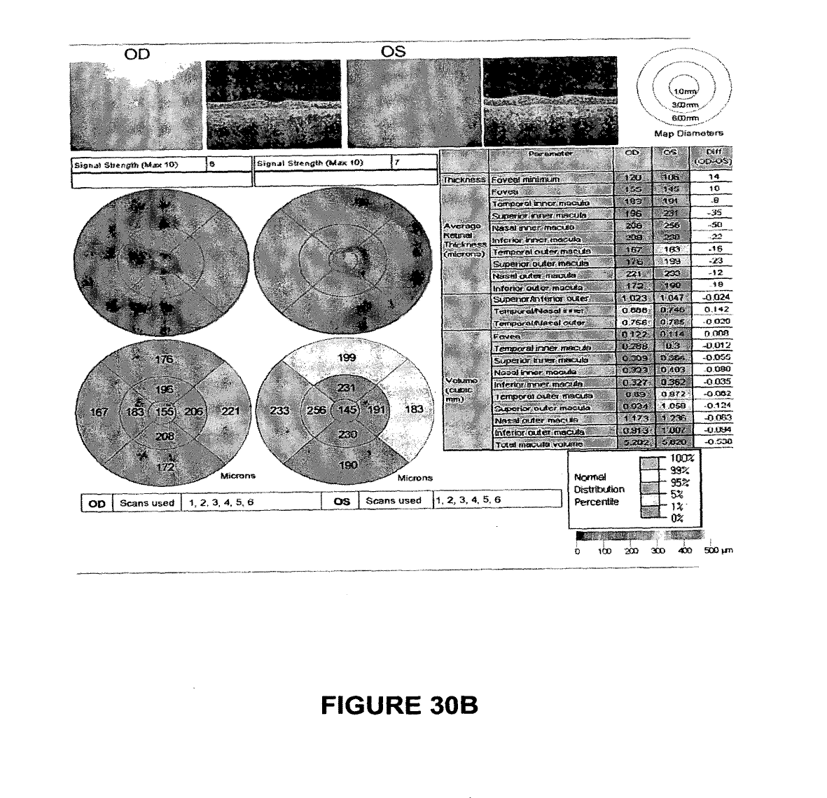

Patient K.T presented with a three-month history of decreased vision in both eyes. A fluoro-angiographic image showed choroid occult new vessel (CNV) with pigmentary epithelium detachment (FIG. 29A). An OTC exam revealed a macular thickening and a pigmentary epithelium detachment (FIG. 30A). He was treated with corticosteroid+non-steroidal anti-inflammatory agent+neosynephrine. Three months after treatment, the patient's vision improved. Fluoro-angiography revealed a decreased activity of the new vessel (FIG. 29B). A follow up OCT scan (FIG. 30B) demonstrated a complete regression of macular thickening and recovery of foveal contour.

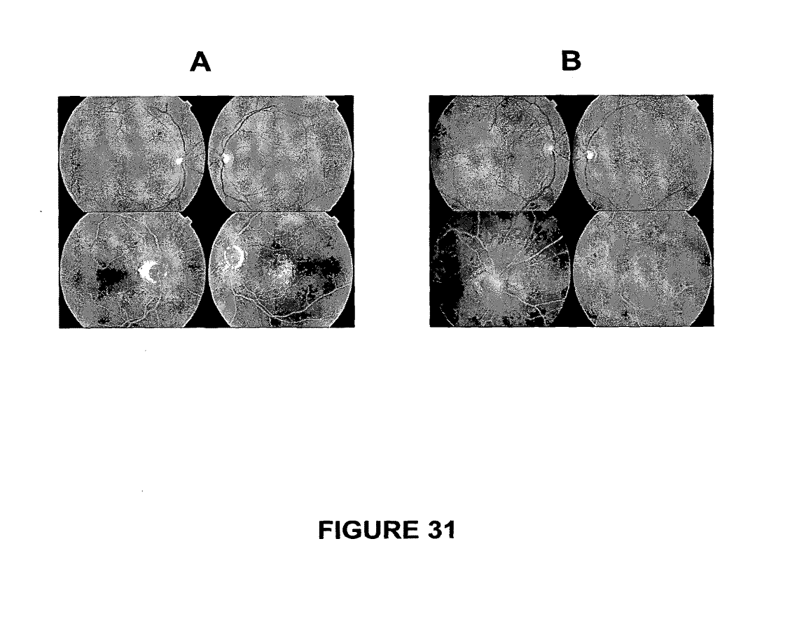

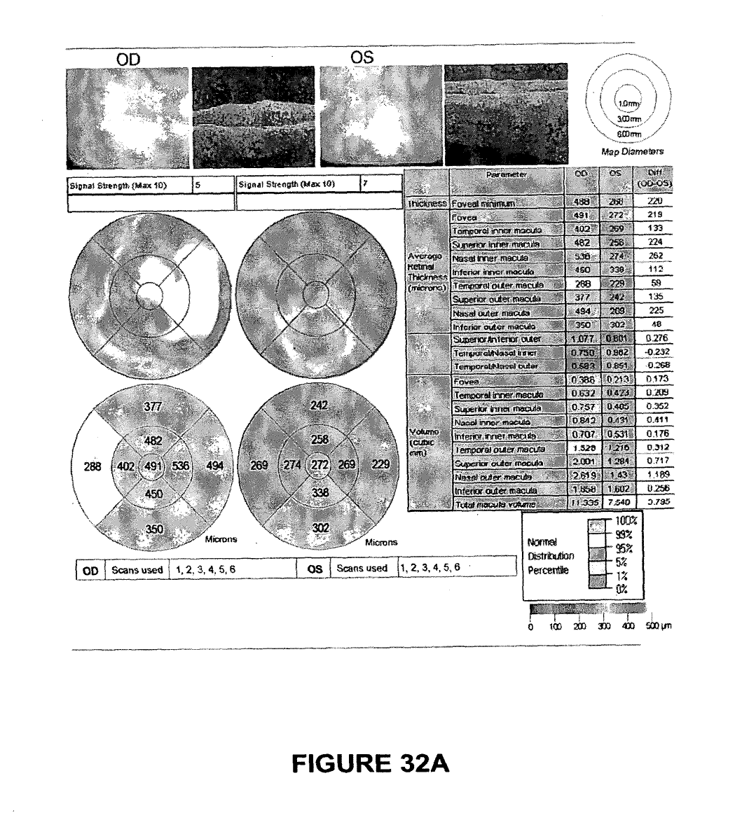

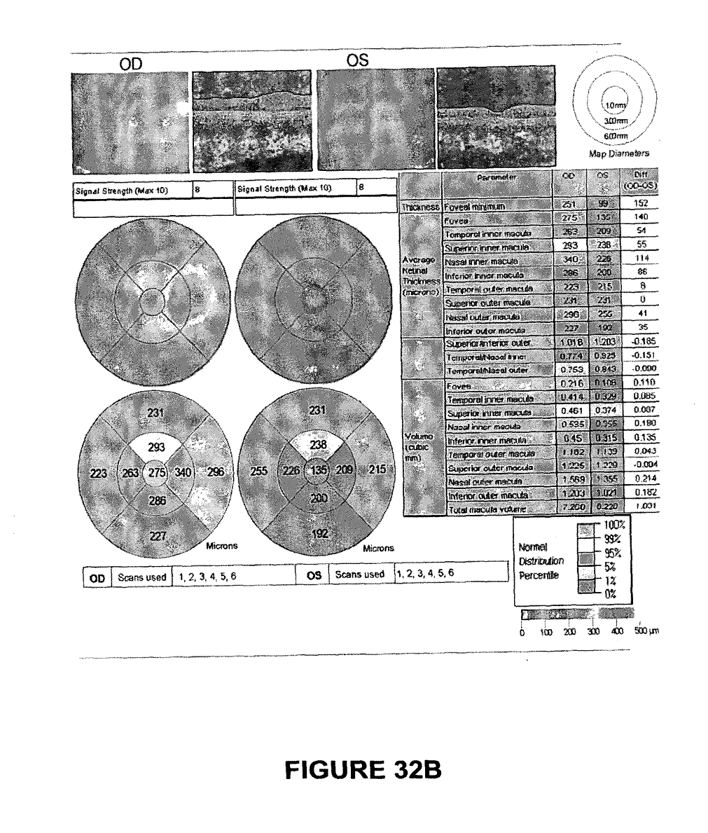

Patient D.F presented with a history of decreased vision in the left eye. A fluoro-angiographic image showed choroid occult new vessel (CNV) (FIG. 31A). Prior to receiving the treatment an OCT scan was taken, which is shown in FIG. 32A. She was treated with corticosteroid+non-steroidal anti-inflammatory agent+apraclonidine. Three months after treatment, the patient's vision improved. Fluoro-angiography revealed a decreased activity of the new vessel (FIG. 31B). Follow up OCT (FIG. 32B) demonstrated a complete regression of macular thickening and recovery of foveal contour.

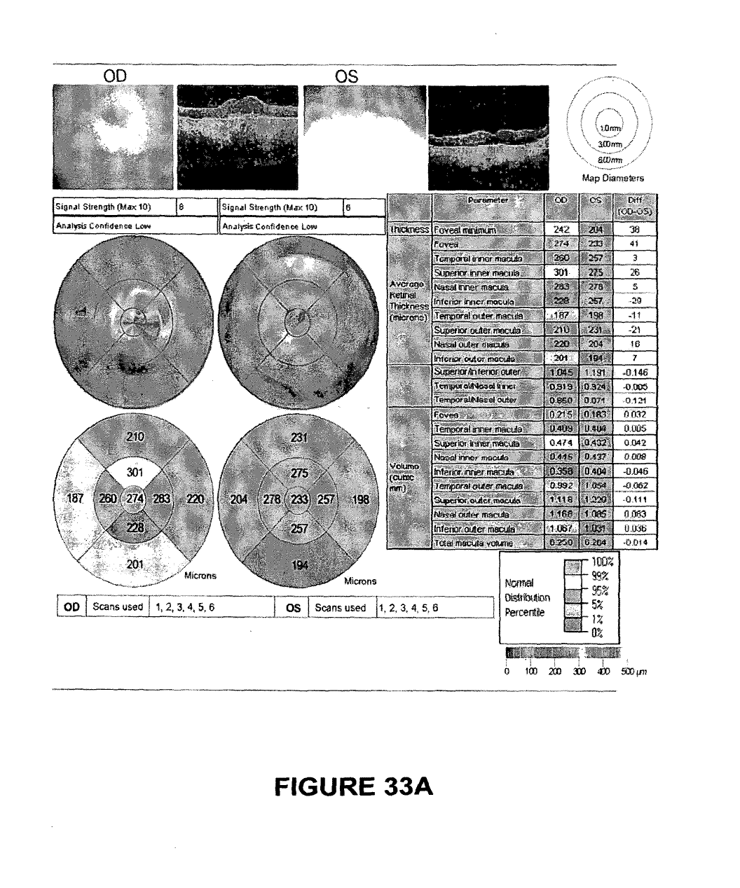

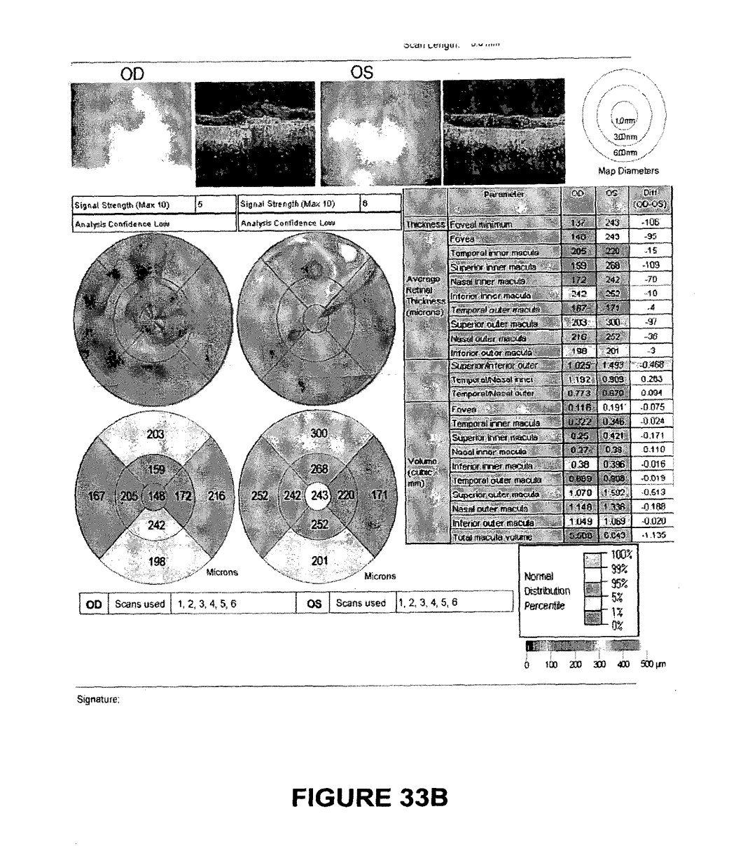

Patient D.M presented with a history of decreased vision in the right eye. A fluoro-angiographic image showed choroid occult new vessel (CNV) (image not shown). Prior to receiving the treatment an OCT scan was taken, which is shown in FIG. 33A. He was treated with Avastin® (anti-VGEF)+apraclonidine. One month after treatment, the patient's vision improved. Fluoro-angiography revealed a decreased activity of the new vessel (image not shown). Follow up OCT (FIG. 33B) demonstrated a complete regression of macular thickening and recovery of foveal contour.

As can be ascertained from the above data the patients improved their vision. Fluoresceinangiography and OCT showed a decrease of choroidal new vessels and macular thickening.

### Example 5

### Miscellaneous Macular Degenerations

In addition to age-related macular degeneration (ARMD), numerous conditions have been associated with the development of chroidal neovascularization (CNV). These conditions, which primarily affect younger patients, include pathologic myopia.

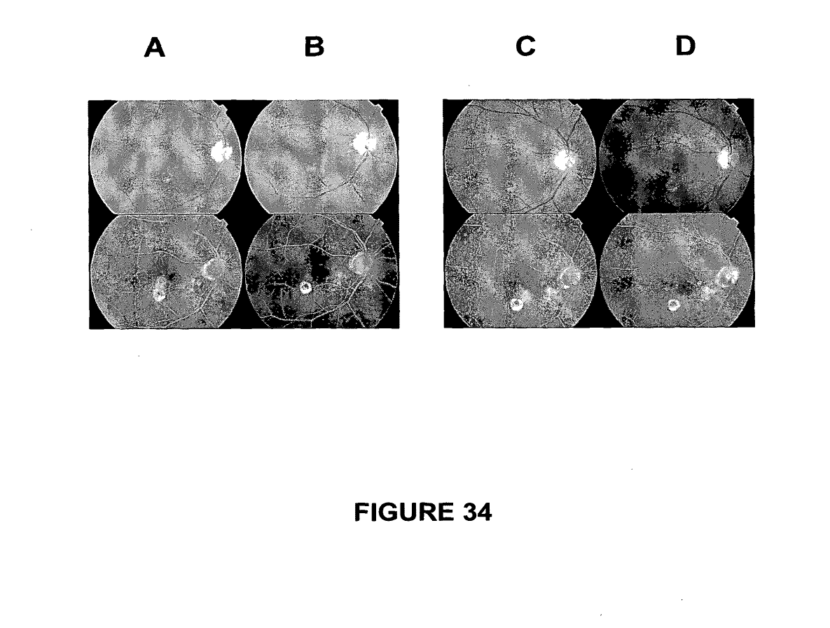

Patient B.S.S. is a thirty year old male who had complaints of decreased central vision in the right eye. Fluorescence-angiography showed early hyperfluorescence with late leakage consistent with subfoveal classic CNV (choroid new vessel) (FIG. 34A). He was treated topically with Avastin® (anti-VGEF)+apraclonidine. Two months later (FIG. 34B), visual acuity improved and new vessel activity regressed. Four months later and because he stopped the treatment (FIG. 34C) a decreased visual acuity associated with recurrence of new vessel activity was observed. He received topically Avastin® (anti-VGEF)+apraclonidine. Two weeks later (FIG. 34D) visual acuity improved and new vessel activity stopped.

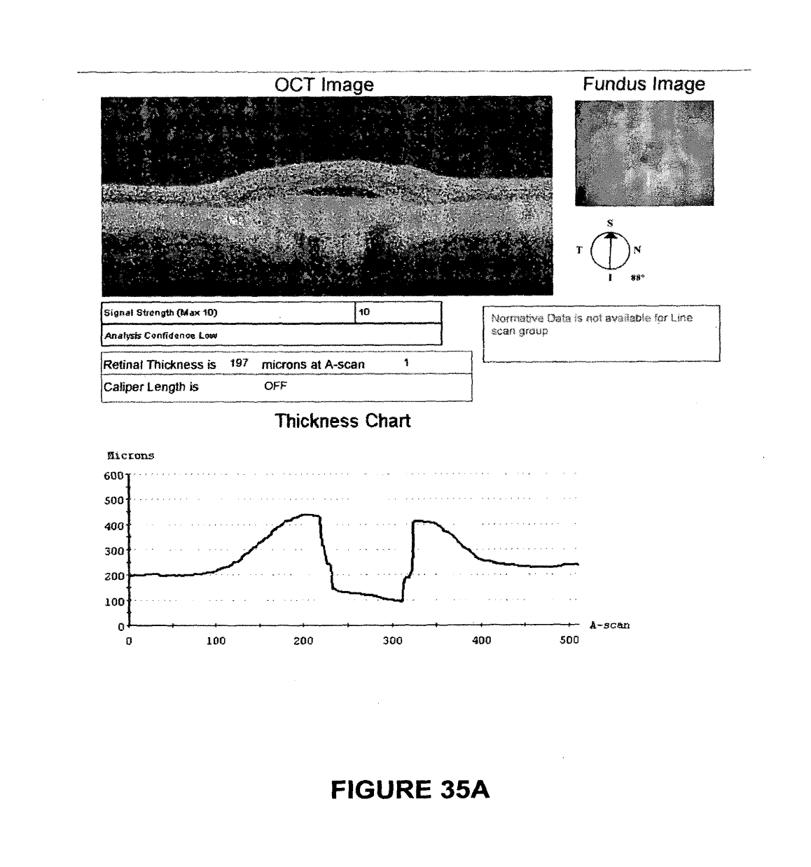

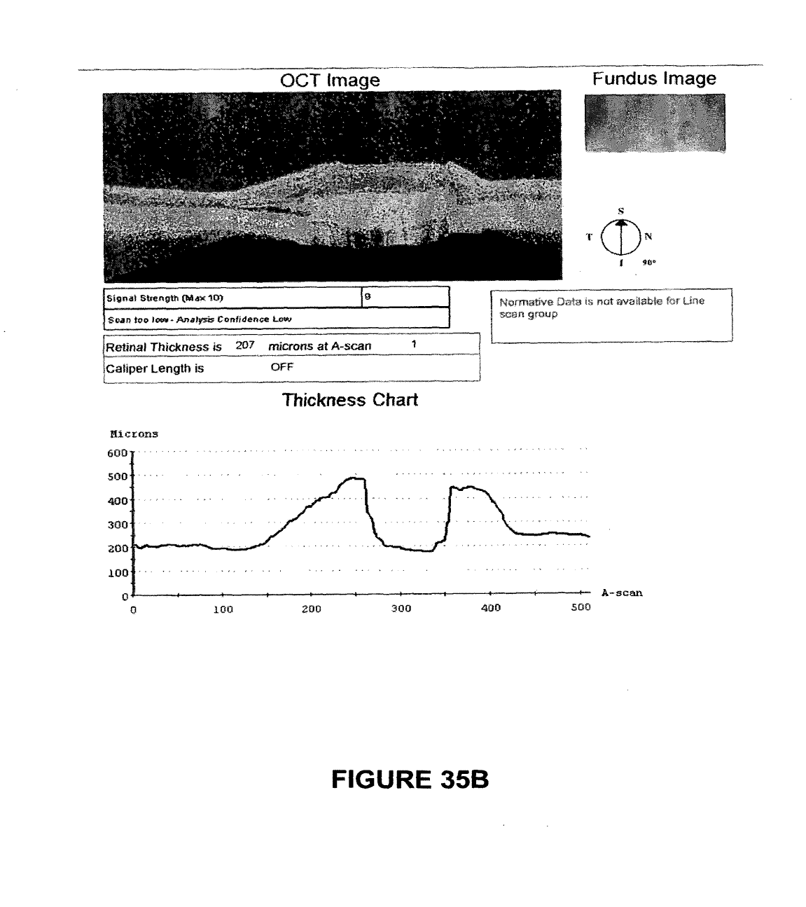

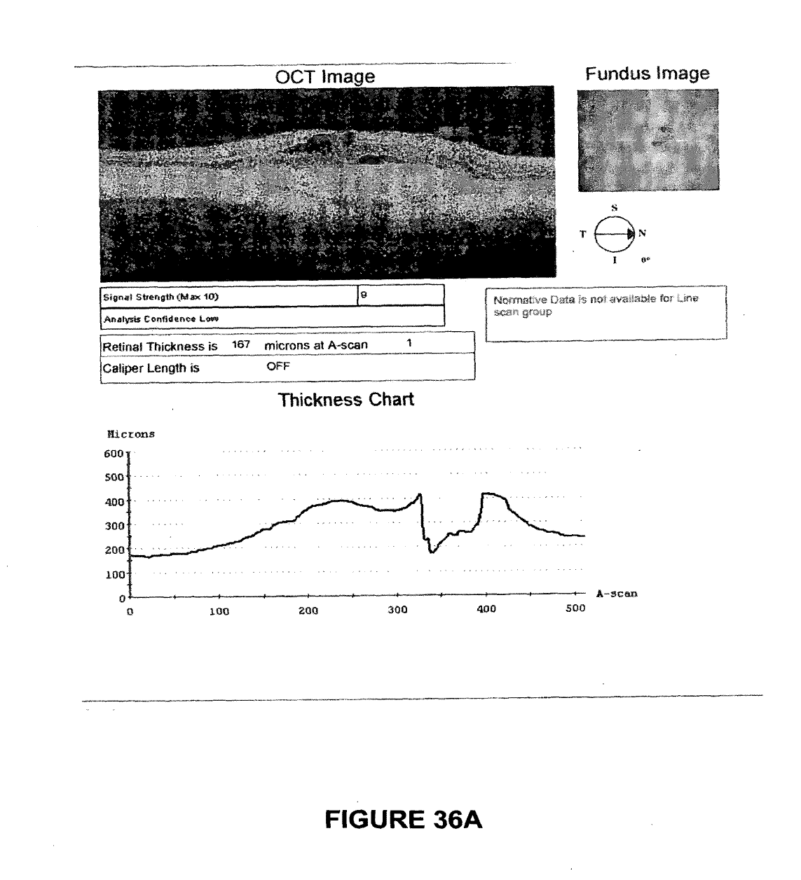

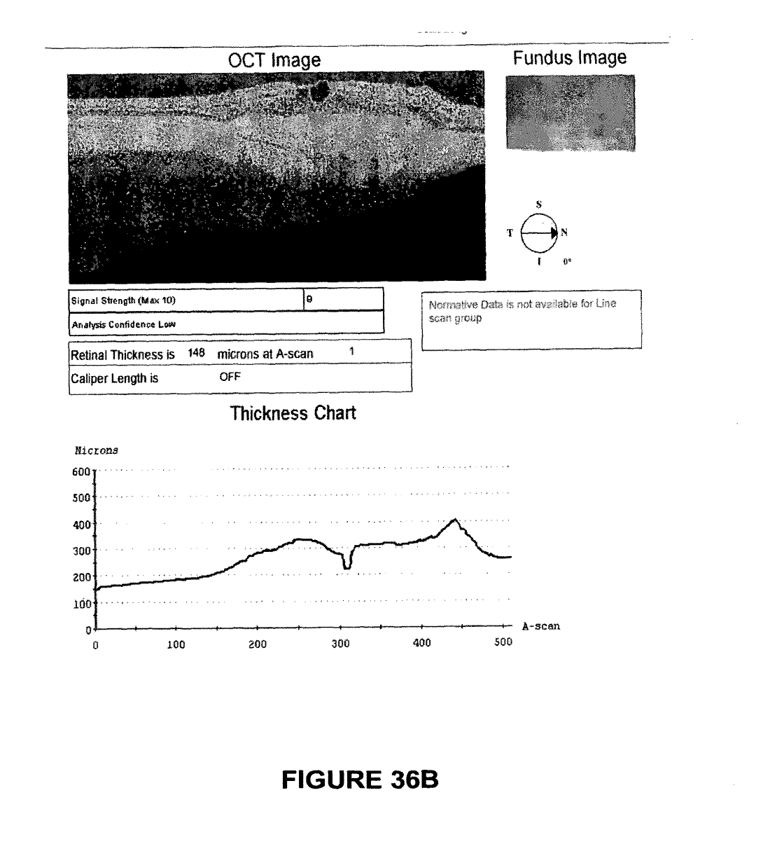

Patient G.C is a twenty nine year old female who had complaints of decreased central vision in the right eye because of classic macular new vessels. The OCT tomogram (FIGS. 35A and 36A) showed an oval hypereflective lesion. There was a pocket of subretinal fluid adjacent to this lesion and mild overlying retinal edema. She was treated with ramipril (Angiotensin converting enzyme inhibitor)+timolol+neosynephrine. One week later visual acuity improved and resolution of the retinal edema at the OCT control was noted (FIGS. 35B and 36B).

### Example 6

### Glaucoma Neuropathy

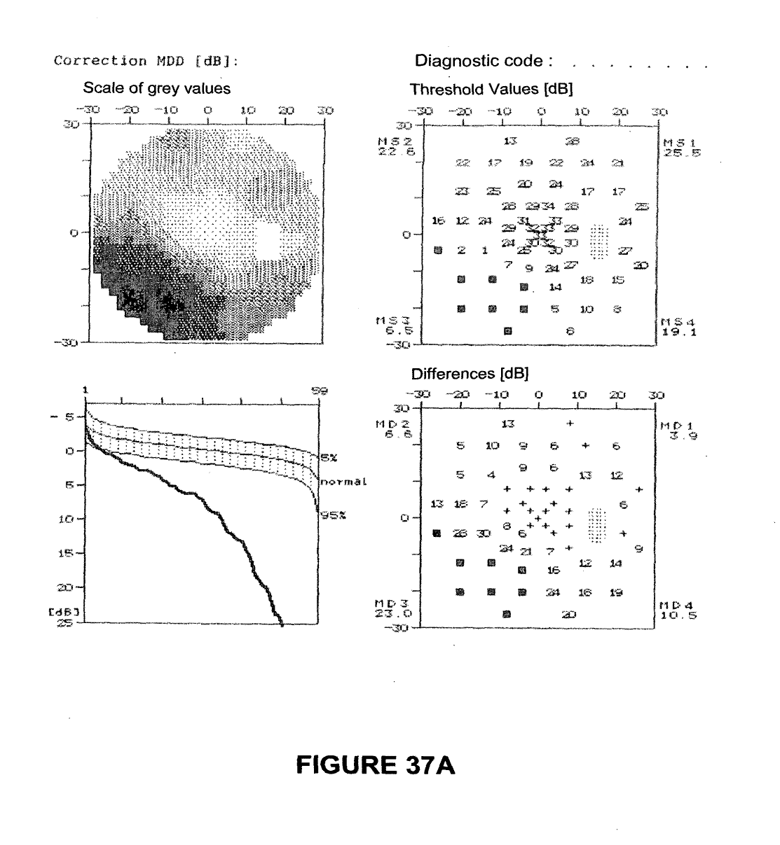

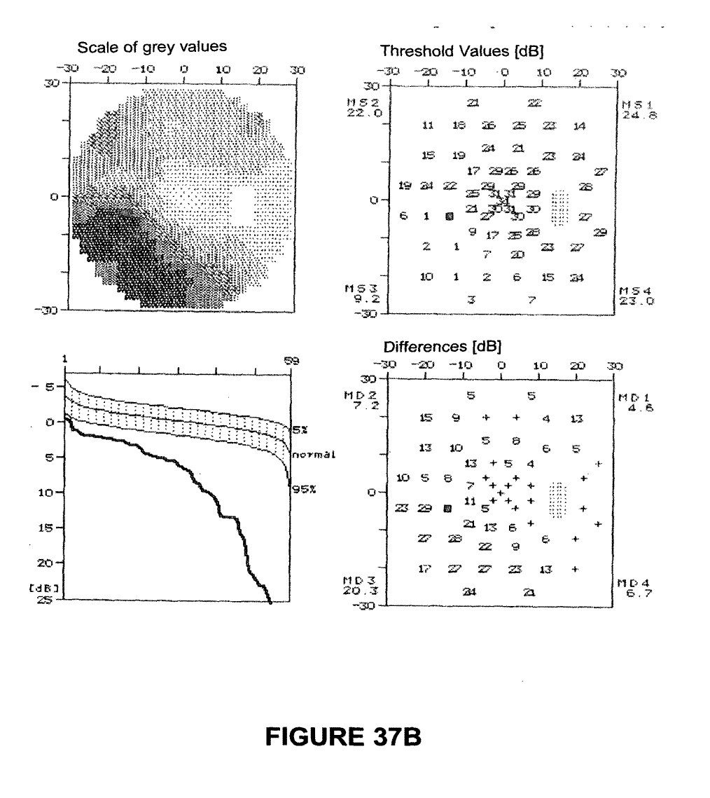

Patient A.L presented with an open angle chronic glaucoma in the right eye. Despite surgical normalization of her intraocular pressure she continued to deteriorate in her field of vision (FIG. 37A). She received topically ramipril (Angiotensin converting enzyme inhibitor)+brimonidine. Two weeks later we noted an improvement of nasal scotoma (FIG. 37B).

In summary of these results showed that the use of a carrier drug such as an alpha adrenergic agonist agent and/or a beta-blocking agent alone or in combination resulted in effective delivery of drugs to treat eye disorders and/or eye diseases.

While the invention has been described in terms of various preferred embodiments, the skilled artisan will appreciate that various modifications, substitutions, omissions and changes may be made without departing from the scope thereof. Accordingly, it is intended that the scope of the present invention be limited by the scope of the following claims, including equivalents thereof.

1.-51. (canceled)

52. A method for delivering drugs to the chorio-retina and optic nerve head of the eyes to treat glaucoma comprising: topically administering at the surface of the eye a drug comprising an effective amount of an angiotensin converting enzyme inhibitor and physiologically acceptable amount of an alpha adrenergic agonist agent, a beta blocking agent and mixtures thereof, in an opthamologically acceptable carrier.

53. The method according to claim 52, wherein the angiotensin converting enzyme is ramipril.

54. The method according to claim 52, wherein the alpha adrenergic agonist agent is selected from the group of neosynephrine, brimonidine, aprachlonidine, clonidine, methoxamine, methylnorepinephrine, oxymetazoline, phenylphrine, pivalat, beta-methylepinephrine, guanfacine, guanabenz, guanoxabenz, guanethidine, tizanidine and mixtures thereof.

55. The method of claim 52, wherein the beta-blocking agent is selected from the group of timolol, sotalol, propranolol, penbutolol, nadolol, metoprolol, labetalol, esmolol, cartelol, bisoprolol, betaxolol, atenolol, acebutolol, levobunolol, metipranolol and mixtures thereof.

56. The method according to claim 52, wherein the beta-blocking agent thereof contacts the eye prior to the effective amount of said angiotensin converting enzyme inhibitor.

57. The method according to claim 52, wherein the alpha adrenergic agent and the beta-blocking agent contacts the eye prior to said effective amount of said angiotensin converting enzyme inhibitor.

58. A method of treating glaucoma comprising:
(a) topically administering to the chorio-retina and optic nerve head of an eye to a person or an animal in need of such treatment a drug comprising an effective amount of an angiotensin converting enzyme inhibitor and a physiologically acceptable amount of an alpha adrenergic agonist agent, a beta-blocking agent and mixtures thereof in an opthamologically acceptable carrier.

59. The method according to claim 58, wherein the effective amount of said angiotensin converting enzyme inhibitor is administered at a concentration of from 0.001 to 15% (w/v).

60. The method according to claim 58, wherein the physiological acceptable amount of said alpha adrenergic agonist agent, a beta-blocking agent and mixtures thereof is administered at a concentration of from 0.01% to 20% (w/v).

61. The method according to claim 59, wherein the angiotensin converting enzyme is ramipril.

62. The method according to claim 60, wherein the alpha adrenergic agonist agent is selected from the group of neosynephrine, brimonidine, aprachlonidine, clonidine, methoxamine, methylnorepinephrine, oxymetazoline, phenylphrine, pivalat, beta-methylepinephrine, guanfacine, guanabenz, guanoxabenz, guanethidine, tizanidine and mixtures thereof.

63. The method of claim 60, wherein the beta-blocking agent is selected from the group of timolol, sotalol, propranolol, penbutolol, nadolol, metoprolol, labetalol, esmolol, cartelol, bisoprolol, betaxolol, atenolol, acebutolol, levobunolol, metipranolol and mixtures thereof.

64. The method according to claim 60, wherein the beta-blocking agent thereof contacts the eye prior to the effective amount of said angiotensin converting enzyme inhibitor.

65. The method according to claim 58, wherein the alpha adrenergic agent and the beta-blocking agent contacts the eye prior to said effective amount of said angiotensin converting enzyme inhibitor.

66. A method of treating glaucoma comprising:
(a) topically administering to the chorio-retina and optic nerve head of an eye to a person or an animal in need of such treatment a drug comprising an effective amount of ramipril and a physiologically acceptable amount of an alpha adrenergic agonist agent, a beta-blocking agent and mixtures thereof in an opthamologically acceptable carrier.

67. A method of treating glaucoma comprising:
(a) topically administering to the chorio-retina and optic nerve head of an eye to a person or an animal in need of such treatment a drug comprising an effective amount of rampiril and a physiologically acceptable amount of brimonidine, timolol and mixtures thereof in an opthamologically acceptable carrier.

68. A method for delivering drugs to the chorio-retina and optic nerve head of the eyes to treat glaucoma comprising: topically administering at the surface of the eye a drug comprising an effective amount of an angiotensin converting enzyme inhibitor and physiologically acceptable amount of an alpha adrenergic agonist agent, or a beta blocking agent, in an opthamologically acceptable carrier.
